(19) United States
(12) Patent Application Publication
(10) Pub. No.: US 2014/0256623 A1
(43) Pub. Date: Sep. 11, 2014

(54) TRANSMUCOSAL DELIVERY OF PHARMACEUTICAL ACTIVE SUBSTANCES

(71) Applicant: ANYGEN CO., LTD., Gwangju (KR)

(72) Inventors: Sangyong JON, Gwangju (KR); EUNHYE LEE, Gwangju (KR); JIN JU LEE, Gwangju (KR); IN-HYUN LEE, Gwangju (KR)

(73) Assignee: ANYGEN CO., LTD., Gwangju (KR)

(21) Appl. No.: 14/286,969

(22) Filed: May 23, 2014

Related U.S. Application Data
(62) Division of application No. 11/847,237, filed on Aug. 29, 2007, now abandoned.

(51) Int. Cl. A61K 47/48; A61K 9/00

(52) U.S. Cl. CPC A61K 47/4823; A61K 9/006; A61K 9/0043; A61K 9/0034; A61K 9/0053; A61K 9/0048
USPC 514/5.9; 514/11.9

(57) ABSTRACT

Provided is a conjugate including a pharmacologically active substance covalently bound to chitosan or its derivative and a method for transmucosal delivery of a pharmacologically active substance using the same. Specifically a conjugate includes a pharmacologically active substance covalently bound via a linker to chitosan; and a pharmaceutical composition for transmucosal administration of a drug includes the aforementioned conjugate and a pharmaceutically acceptable carrier. Further provided is a method for in vivo delivery of a pharmacologically active substance via a transmucosal route, by covalent binding of the active substance with chitosan or its derivative via a linker. The conjugate in accordance with the present invention exhibits excellent absorption rate and biocompatibility in biological mucous membranes, particularly mucous membranes of the alimentary canal (especially the gastrointestinal tract), in vivo degradability, and superior bioavailability even with oral administration, thus enabling treatment of diseases via oral administration of a drug.

### CROSS-REFERENCES TO RELATED APPLICATIONS

This is a continuation-in-part of International application PCT/KR2007/000403, which was filed with the Republic of Korea Receiving Office on Jan. 23, 2007, and claims the benefit of priority of Republic of Korea applications KR 10-2006-0006632 filed Jan. 23, 2006, KR 10-2006-0068801 filed Jul. 22, 2006, and KR 10-2006-0068804 filed Jul. 22, 2006. The benefit of priority is claimed to each of the above-noted Republic of Korea applications, which—together with the above-referenced International application—are incorporated by reference herein.

### FIELD OF THE INVENTION

The present invention relates to a conjugate including a pharmacologically active substance covalently bound to chitosan or its derivative and a method for transmucosal delivery of a pharmacologically active substance using the same.

### BACKGROUND

With great advances in genetic engineering and bioprocess technologies, it has become possible to achieve industrial-scale production of various peptides and protein drugs, e.g., biopharmaceutical products (hereinafter also referred to as “biodrugs”), which have suffered from difficulties in chemical synthesis. However, most proteins exhibit non-absorptive tendencies through the mucous membranes of animals due to huge molecular weight and specific molecular structure, thereby suffering from difficulty in application thereof for oral preparations. Therefore, an administration route of proteins is confined to injection, which is accompanied by various problems such as difficulty of medication upon chronic administration of drugs and fear and rejection of injection therapy to patients. Therefore, development of oral preparations having no burden of injection administration on patients by increasing an enteric absorption rate of biodrugs will obviate fear and rejection of injection and enables the patients to easily take a drug in compliance with medication instructions, thereby leading to improvements in the short- and long-term life quality of patients.

For these reasons, various attempts have been actively made to enhance in vivo stability and absorption rate of therapeutic proteins. Among such trials, the most well-known approach is PEGylation, the process by which polyethylene glycol (PEG) chains are chemically attached to proteins or peptides. At the early stage of introduction, this technique was used to reduce antigenicity of target materials. Now, PEGylation is largely employed for improvement of in vivo stability and absorption rate of target proteins by increasing an in vivo residence time of the proteins.

In addition to PEGylation, a great deal of research has been focused lately on a method of using the biodrug in conjunction with a substance that is capable of enhancing the permeability of an intestinal epithelial cell membrane, such as a fatty acid, a bile acid and the like, a method of using a substance (for example, Vitamin B12 and Fc receptor) that is capable of selectively binding to a receptor of the intestinal epithelial cell membrane, a method of increasing a drug absorption rate from the intestinal mucosa via a conjugate of insulin with a fat-soluble substance including lipid and bile acid through the direct chemical bonding therebetween, a method of drug delivery by inclusion of protein drugs into microparticles or nanoparticle of biodegradable polymers.

However, these methods still have disadvantages such as very low in vivo absorption and bioavailability upon oral administration and potential safety risk due to the use of additives in conventional formulations for oral applications that may exhibit toxicity upon chronic administration.

In recent years, there has been a great deal of interest in developing a method for delivery of an anti-cancer drug that is poorly water-soluble, particularly paclitaxel, an anti-neoplastic agent effective against a wide range of cancers including breast cancer and ovarian cancer. Meanwhile, paclitaxel has a very low solubility in conventional aqueous vehicles including water and therefore is formulated into a vehicle containing ethanol and Cremophor EL. For this reason, administration of the anti-cancer drug paclitaxel via intravenous infusion causes severe side effects such as hypersensitivity reactions. In order to overcome such shortcomings of paclitaxel therapy a variety of attempts have been made including micellular formulation, conjugation with a variety of water-soluble macromolecules, and prodrug approaches. Meanwhile, with the increases in such research and study, there has been a great deal of focus in recent years on development of an oral delivery system for paclitaxel. This is because such an oral formulation of paclitaxel is preferable for treatment of chronic diseases including cancers and is greatly beneficial fur patients by providing easy and convenient administration without a need to go to the hospital for an intravenous infusion. However oral administration of paclitaxel poses a disadvantage of low bioavailability. According to recent research publication reports in scientific articles and journals, the bioavailability of paclitaxel was increased to a clinically valuable level. One of the those research papers reported that the combined use of paclitaxel with a P-glycoprotein (P-gp) inhibitor such as cyclosporin A (CsA) or Valspodar resulted in a very high increase in the bioavailability of paclitaxel (ca. 50-60% vs. ca. 4-10% with PTX only). Despite such a favorable result, P-gp is known to protect the gastrointestinal tract, cerebrum and excretory organs against xenotoxin and therefore use of the P-gp inhibitor may potentially cause adverse side effects. Furthermore, other studies were reported including methods of preparing emulsions of paclitaxel using surfactants and methods of encapsulating paclitaxel into biodegradable polymer nanoparticles. However, use of excessive amounts of surfactants may bring about toxicity to the subjects, and the above methods have the drawback of low bioavailability.

Therefore, there is a strong need for the development of a pharmaceutical formulation that can provide administration of an anti-cancer drug, such as paclitaxel, via an oral route capable of exhibiting high bioavailability of the drug.

Transmucosal delivery is a method for administration of pharmacologically-active substances and provides great advantages. Owing to the ability of transmucosal delivery to achieve systemic and local drug effects on target sites, the transmucosal delivery system has received a great deal of attention as an attractive drug delivery system that can cope with specific regimens of drugs. Transmucosal delivery not only rapidly exerts therapeutic effects but also exhibits rapid drug clearance, consequently increasing bioavailability of the drug. In addition, the transmucosal delivery system is superior with respect to patient medication compliance, as compared to other administration methods.

Due to the aforementioned advantages of the transmucosal delivery system, many efforts have been made to develop more advanced transmucosal delivery systems. International Publications No. WO 2005/032554 and WO 2005/016321, and U.S. Pat. Nos. 6,896,519, 6,564,092 and 6,506,730 (each of which is incorporated herein by reference) relate to transmucosal delivery systems. In addition, U.S. patent application Ser. No. 07/579,375 (issued as U.S. Pat. No. 5,194,594, which is incorporated herein by reference) discusses antibodies which have been modified by chemical conjugation with succinimidyl 3-(2-pyridyldithio)propionate (SPDP), and U.S. patent application Ser. No. 08/167,611 (issued as U.S. Pat. No. 5,554,388, which is incorporated herein by reference) discusses a composition for administration to the mucosa including a pharmacologically active compound and a polycationic substance. Also, U.S. Pat. No. 6,913,746 (which is incorporated herein by reference) describes complexes consisting of immunoglobulins and polysaccharides for oral and transmucosal use, and U.S. Patent Application No. 2005/0175679 A1 (which is incorporated herein by reference) describes a composition for transmucosal administration, including morphine and a water-soluble polymer.

However, most attempts to develop methods capable of achieving oral administration of protein drugs or anticancer drugs via transmucosal delivery of drugs were found futile, with few successful results, and resulting in unsatisfactory therapeutic efficacy of drugs.

### SUMMARY

The inventors of the present invention have performed intensive research to develop a drug delivery system that can realize transmucosal delivery, particularly oral transmucosal delivery of drugs while overcoming side effects and disadvantages suffered by conventional drug delivery systems of pharmacologically active substances. Surprisingly the present inventors have discovered that it is possible to elicit excellent pharmacological efficacy of desired drugs in vivo by utilizing a mucoadhesive polymer, exhibiting an excellent in vivo mucosal absorption rate, safety and in vivo degradability, as a delivery system capable of achieving the above-mentioned purposes, and oral administration of a conjugate including a pharmacologically active substance covalently bound to the mucoadhesive polymer.

Accordingly, the present invention has been made in view of the above (and various other) problems, and it is in accordance with one aspect of the present invention to provide a conjugate including a pharmacologically active substance and chitosan or its derivative covalently bound to each other via a linker.

It is in accordance with another aspect of the present invention to provide a pharmaceutical composition for transmucosal administration of a drug, including the aforementioned conjugate and a pharmaceutically acceptable carrier.

It is in accordance with still another aspect of the present invention to provide a method for in vivo delivery of a pharmacologically active substance via a transmucosal route, by covalent binding of the active substance with a mucoadhesive polymer via a linker.

It is in accordance with a further aspect of this invention to provide a method for increasing the transmucosal absorption of a pharmacologically active substance of which transmucosal absorption is inhibited by P-glycoprotein.

Other objects and advantages of the present invention will become apparent from the detailed description set forth below, together with the appended claims and drawings.

### BRIEF DESCRIPTION OF THE DRAWINGS

FIG. 1 is a graph showing changes in the relative blood glucose levels of animals after intravenous injection of an insulin-chitosan conjugate of the present invention into the tail veins of diabetes-induced male rats (“i.v.” denotes intravenous injection; “s.c.” denotes subcutaneous injection; and “Insulin-6K LMWC” denotes an insulin-6 KDa low molecular weight chitosan conjugate).

FIG. 2 is a graph showing changes in the relative blood glucose levels of animals after oral administration of an insulin-chitosan conjugate solution to diabetes-induced male rats, in accordance with an embodiment of the present invention.

FIG. 3 is a graph showing activities of salmon calcitonin-chitosan conjugates in accordance with the present invention (“sCT” denotes salmon calcitonin).

FIG. 4 represents calcitonin levels in blood after oral administration of calcitonin-chitosan conjugates to rats.

FIGS. 5a and 5b are graphs showing results of MTT assay for cytotoxic effects of a paclitaxel-chitosan conjugate on tumor cells, in accordance with the present invention, in which FIG. 5a related to B16F10 murine melanoma, and FIG. 5b relates to MDA-MB-231 human breast carcinoma (“PTK” denotes paclitaxel).

FIGS. 6a and 6b represent effects of P-glycoprotein (P-gp) inhibitor after oral administration of paclitaxel and paclitaxel-chitosan (MW: 6000) conjugates in vivo.

FIG. 7 is a graph showing analysis results of allograft experiments for in vivo anti-cancer effects of a paclitaxel-chitosan conjugate in accordance with the present invention.

FIG. 8 is a graph showing a survival rate of animals after oral administration of a paclitaxel-chitosan conjugate to mice.

FIG. 9 represents in vivo anti-tumoric effects of anticancer agent-chitosan conjugates in Accordance with the present invention, in which the anticancer agents linked to chitosan include docetaxel, doxorubicin and camptothecin.

### DETAILED DESCRIPTION

In accordance with one aspect of the present invention, there is provided a conjugate for transmucosal delivery comprising a pharmacologically active substance covalently bound via a linker to chitosan or its derivative.

In accordance with another aspect of the present invention, there is provided a pharmaceutical composition for transmucosal administration of a drug, comprising the aforementioned conjugate and a pharmaceutically acceptable carrier.

In still another aspect of the present invention, there is provided a method for in vivo delivery of a pharmacologically active substance via a transmucosal route, which includes preparing a conjugate by binding covalently the pharmacologically active substance to chitosan or its derivative via a linker, and administering the conjugate to a subject via the transmucosal route.

The conjugate includes two essential components: pharmacologically active substances, and chitosan or its derivative as mucoadhesive polymers.

As used herein the term “pharmacologically active substance” refers to any material having a desired pharmacological activity including proteins, peptides and chemicals. The pharmacologically active substance may include recombinantly or synthetically prepared substances and/or other substances isolated from natural sources. As used herein the term “protein” refers to a polymer of amino acids in peptide linkages and the term “peptide” refers to an oligomer of amino acids in peptide linkages.

As examples, the proteins or peptides that may used as the pharmacologically active substance in the present invention may include (but is not limited to) hormones, hormone analogues, enzymes, enzyme, inhibitors, signaling proteins or fragments thereof, antibodies or fragments, single-chain antibodies, binding proteins or binding domains thereof, antigens, attachment proteins, structural proteins, regulatory proteins, toxin proteins, cytokines, transcriptional regulatory factors and/or blood coagulation factors. Preferably, the pharmacologically active substance of the present invention may include materials that can be used as a protein drug, for example insulin, insulin-like growth factor 1 (IGF-1), growth hormones, interferons (IFNs), erythropoietins, granulocyte-colony stimulating factors (G-CSFs), granulocyte/macrophage-colony stimulating factors (GM-CSFs), interleukin-2 (IL-2), epidermal growth factors (EGFs), calcitonin, adrenocorticotropic hormone (ACTH), atobisban, buserelin, cetrorelix, deslorelin, desmopressin, dynorphin A (1-13), elcatonin, eleidosin, eptifibatide, GHRH-II (growth hormone releasing hormone-II), gonadorelin, goserelin, histrelin, leuprorelin, lypressin, octreotide, oxytocin, piressin, secretin, sincalide, terlipressin, thymopentin, thymosine α1, triptorelin, bivalirudin, carbetocin, cyclosporin O, exedine, lanreotide, LHRH (luteinizing hormone-releasing hormone), naferelin, parathyroid hormone, pramlintide, T-20 (enfuvirtide), thymalfasin and/or ziconotide. More preferred is insulin, IGF-1 or calcitonin. Most preferred is insulin.

Further, the pharmacologically active substance of the present invention may include any anti-cancer drug that is used as an anti-cancer chemotherapeutic agent, for example, preferably cisplatin, carboplatin, procarbazine, mechlorethamine, cyclophosphamide, ifosfamide, melphalan, chlorambucil, bisulfan, docetaxel, camptothecin, nitrosourea, dactinomycin (actinomycin-D), daunorubicin, doxorubicin, bleomycin, plicomycin, mitomycin, etoposide, tamoxifen, paclitaxel, transplatinum, 5-fluorouracil, adriamycin, vincristine, vinblastine and/or methotrexate. More preferably, the anti-cancer drug delivered by conjugates of this invention is paclitaxel, docetaxel, doxorubicin or camptothecin, and most preferably paclitaxel.

According to a preferred embodiment, the pharmacologically active substance is a chemical drug of which transmucosal absorption is inhibited by P-glycoprotein. Surprisingly, the present inventors have found that the chitosan conjugate in accordance with the present invention overcomes the shortcomings associated with the inhibition of the transmucosal absorption of drugs by P-glycoprotein.

More preferably, the chemical drug whose transmucosal absorption is inhibited by P-glycoprotein is a hydrophobic drug. Still more preferably, the chemical drug useful in this invention includes anti-cancer drugs such as cisplatin, methotrexate, paclitaxel, daunorubicin, doxorubicin, vincristine, vinblastine, carboplatin, procarbazine, mechlorethamine, cyclophosphamide, ifosfamide, melphalan, chlorambucil, bisulfan, docetaxel, camptothecin, nitrosourea, dactinomycin (actinomycin-D), bleomycin, plicomycin, mitomycin, etoposide, tamoxifen, transplatinum, 5-fluorouracil, adriamycin, quinolone, ciprofloxacin, progesterone, teniposide, estradiol, epirubicin and/or taxanes; prostaglandins; amphotericin B; Vitamin E; steroids such as testosterone, beclomethasone, cortisone, dexamethasone, triamicinolone, aldosterone, methylprednisolone and/or betamethasone valerete; antiepileptic drugs such as phenytoin; antidepressant such as citacitalopram, thioperidone, trazodone, trimipramine, amitriptyline and/or phenothiazines; antipsychotic drugs such as fluphenazine, haloperidol, thioridazine, and/or trimipramine; protease inhibitors such as amprenavir, indinavir, lopinavir, nelfinavir, ritonavir and/or saquinavir; calcium blockers such as bepridil, diltiazem, flunarizine, lomenzine, secoverine, tamolarizine, verapamil, nicardipine, prenylamine and/or fendiline; and/or cardiac drugs such as digoxin, diltiazem, verapamil and/or talinolol.

### Mucoadhesive Polymers

As used herein, the term “mucoadhesive polymer” refers to a polymer having a good in vivo mucosal absorption rate, safety and degradability. The mucoadhesive polymer used in the present invention may be synthesized or may be naturally-occurring materials.

Examples of naturally-occurring mucoadhesive polymers may include, but are not limited to, chitosan hyaluronate, alginate, gelatin collagen, and/or derivatives thereof. Examples of synthetic mucoadhesive polymers may include, but are not limited to, poly(acrylic acid), poly(methacrylic acid), poly(_{L}-lysine), poly(ethylene imine), poly(2-hydroxyethyl methacrylate), and/or derivatives or copolymers thereof.

Most preferably, the mucoadhesive polymer of the present invention is chitosan or its derivative. Chitosan may be prepared by deacetylation of chitin. Next to cellulose, chitin is one of the most abundant organic polymers in nature, with as much as ten billion tons of chitin and its derivatives estimated to be produced from living organisms each year. Chitin is quantitatively found in the epidermis exoskeletons of crustaceans such as crabs and shrimps and insect such as grasshoppers and dragonflies, and in the cell walls of fungi, mushrooms such as Enoki Mushroom (Flammulina velutipes) and Shiitake mushrooms (Lentinus edodes) and bacteria. From a viewpoint of a chemical structure, chitin is a linear polymer of beta 1-4 linked N-acetyl-D-glucosamine units composed of mucopolysaccharides and amino sugars (amino derivatives of sugars). Chitosan is formed by removal of acetyl groups from some of the N-acetyl glucosamine residues (Errington N, et al., “Hydrodynamic characterization of chitosan varying in molecular weight and degree of acetylation,”+Int J Biol Macromol. 15:1123-7 (1993), incorporated herein by reference.) Due to removal of acetyl groups that were present In the amine groups, chitosan is present as polycations in acidic solutions, unlike chitin. As a result, chitosan is readily soluble in an acidic aqueous solution and therefore exhibits excellent processability and relatively high mechanical strength after drying thereof. Due to such physicochemical properties, chitosan is molded into various forms for desired applications, such as powders, fibers, thin films, gels, beads, or the like, depending desired applications (E. Guibal, et al. Ind. Eng. Chem. Res. 37:1454-1463 (1998), incorporated by reference herein). Chitosan is divided into a chitosan oligomer form composed of about 12 monomer units and a chitosan polymer form composed of more than 12 monomer units, depending upon the number of constituent monomer units. In addition, the chitosan polymer is subdivided into three different types, low-molecular weight chitosan (LMWC, molecular weight of less than 150 kDa), high-molecular weight chitosan (HMWC, molecular weight of 700 to 1000 kDa), and medium-molecular weight chitosan (MMWC, molecular weight between LMWC and HMWC).

Due to excellent stability environmental friendliness, biodegradability and Biocompatibility, chitosan is widely used for a variety of industrial and medical applications. Further, it is also known that chitosan is safe and also exhibits no immunoenhancing side effects. The in vivo degradation of chitosan molecules by lysozyme produces N-acetyl-D-glucosamine which is used in the synthesis of glycoproteins and finally excreted fn the form of carbon dioxide (CO₂) (Chandy T, Sharma C P. “Chitosan as a biomaterial,” Biomat Art Cells Art Org. 18:1-24 (1990), incorporated herein by reference).

Chitosan that can be used in the present invention may include any type of chitosan conventionally used in the art, Chitosan of the present invention has a molecular weight of preferably 500 to 20000 Da, more preferably 500 to 15000 Da, still more preferably 1000 to 10000 Da, and most preferably 3000 to 9000 Da. If the molecular weight of chitosan is lower than 500 Da, this may result in poor function of chitosan as a carrier. On the other hand, if the molecular weight of chitosan is higher than 20000 Da, this may lead to a problem associated with formation of self-aggregates in an aqueous solution. The preferred chitosan used in the present invention is oligomeric chitosan.

In conjugates of this invention, chitosan derivatives also may be utilized for transmucosal delivery of drugs. Various chitosan derivatives may be prepared by linking alkyl groups with —OH groups or —NH₂ groups on chitosan. Preferably, the chitosan derivative is an N-chitosan derivative. Suitable alkyl substituents include saturated or unsaturated, branched or unbranched C₁-C₆ alkyl groups such as methyl, ethyl and propyl groups.

### Pharmacologically Active Substance-Chitosan Conjugate

The conjugate of the present invention is characterized in that the pharmacologically active substance and chitosan are covalently bound to each other via a linker. The covalent bonding between the pharmacologically active substance of the present invention and The mucoadhesive polymer may be formed depending open various kinds of bonds. Examples of covalent bonds may include disulfide bonds, peptide bunds, imine bonds, ester bonds and amide bonds. Further, the covalent bonding is formed largely by two types: direct bonding and indirect bonding.

According to the direct bonding method, a covalent bond may be formed by direct reaction of a functional group (for example, —SH, —OH, —COOH, and NH₂) on the pharmacologically active substance with a functional group (for example, —OH and —NH₂) on chitosan. According to the indirect bonding method, the pharmacologically active substance-mucoadhesive polymer complex may be formed by the medium of a compound conventionally used as a linker in the art. In a preferred embodiment, the conjugate of the present invention is covalently bound via the linker.

The linker used in the present invention may be any compound that is conventionally used as a linker in the art. The linker may be appropriately selected depending upon kinds of the functional groups present on the pharmacologically active, substance.

Specific examples of the linker may include, but are not limited to, N-succinimidyl iodoacetate, N-hydroxysuccinimidyl bromoacetate, m-maleimidobenzoyl-N-hydroxysuccinimide ester, m-maleimidobenzoyl-N-hydroxysulfosuccinimide ester, N-maleimidobutyryloxysuccinamide ester, N-maleimidobutyryloxy sulfosuccinamide ester, E-maleimidocaproic acid hydrazide.HCl, [N-(E-maleimidocaproyloxy)-succnamide], [N-(E-maleimidocaproyloxy)-sulfosuccinamide], maleimidopropionic acid N-hydroxysuccinimide ester, maleimidopropionic acid N-hydroxysulfosuccinimide ester, maleimidopropionic acid hydrazide.HCl, N-succinimidyl-3-(2-pyridyldithio)propionate, N-succinimidyl-(4-iodoacetyl) aminobenzoate, succinimidyl-N-maleimidomethyl)cyclohexane-l-carboxylate, succinimidyl-4-(p-maleimidophenyl)butyrate, sulfosoccinimidyl-(4-iodoacetyl)aminobenzoate, sulfosuccinimidyl-4-(N-maleimidomethyl)cyclohexane-1-carboxylate, sulfosuccinimidyl-4-(p-meleimidophenyl)butyrate, m-maleimidobenzoic acid hydrazide.HCl, 4-(N-maleimidomethyl)cyclohexane-l-carboxylic acid hydrazide.HCl, 4-(4-N-maleimidophenyl)butyric acid hydrazide.HCl, N-succinimidyl 3-(2-pyridyldithio)propionate, bis(sulfosuccinimidyl)suberate, 1,2-di[3′-(2′-pyridyldithio)propionamido]butane, disuccinimidyl suberate, disuccinimidyl tartrate, Disulfosuccinimidyl tartrate, dithio-bis-(succinimidylpropionate), 3,3′-dithio-bis-(sulfosuccinimidyl-propionate), Ethylene glycol bis(succinimidylsuccinate) and ethylene glycol Bis(sulfosuccinimidylsuccinate). In a preferred embodiment of the present invention, the covalent bonding of the protein or peptide and chitosan involves interposition of the linker of —CO—(CH₂)ₙ-S—S—(CH₂)ₙ—CO— (Formula I) therebetween. Here, —NH₂ of chitosan and —NH₂ of the protein are respectively bound to the linker via the amide bond. In the Formula I, n is an integer of 1 to 5.

In a specific embodiment of the present invention, the conjugate of the protein or pephde (e.g. insulin) and chitosan has a structure wherein —CO—(CH₂)₂—S—S—(CH₂)₂—CO— is interposed between two components and —NH₂ of chitosan and —NH₂ of the protein are respectively covalently bound to the linker via the amide bond.

Further, in the preferred embodiment of the present invention, covalent bonding of an anti-cancer drug and chitosan involves interposition of a succinyl group therebetween. Here, the succinyl group and chitosan forms an amide bond, and the succinyl group and the anticancer drug forms an aster bond. In a specific embodiment of the present invention, the succinyl group (—CO—CH₂—CH₂—CO—) is interposed between the anti-cancer drug (e.g. paclitaxel) and chitosan, and the succinyl group and chitosan are covalently bound to each other via the amide bond.

The conjugate of the present invention is characterized by being capable of delivering the pharmacologically active substance via transmucosal routes. For example, administration route for transmucosal delivery of the conjugate may include, but are not limited to, mucous membranes of buccal cavity, nasal cavity, rectum vagina urethra, throat, alimentary canal, peritoneum and eyes. The conjugate of the present invention enables oral administration of the drug by delivery of the pharmacologically active substance via a mucous membrane of the alimentary canal.

### Pharmaceutical Composition

In another aspect, the present invention also provides a pharmaceutical composition for transmucosal administration of a drug, comprising a therapeutically effective amount of the conjugate of the present invention and a pharmaceutically acceptable carrier.

As used herein, the term “therapeutically effective amount” refers to ah amount enough to achieve inherent therapeutic effects of the pharmacologically active substance. Also, as used herein, the term “pharmaceutically acceptable” refers to a formulation of a compound that is physiologically acceptable and does not cause allergic response or similar response such as gastric disorder, vertigo, and the like, when it is administered to a human.

The pharmaceutical acceptable carrier may be a material that is conventionally used in preparation of a pharmaceutical formulation. Examples of the pharmaceutically acceptable carrier that can be used in the present invention may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate and mineral oil. Besides the aforesaid ingredients, the pharmaceutical composition of the present invention may further comprise a lubricant, a wetting agent, a sweetening agent, a flavoring agent, an emulsifying agent, a suspending agent, a preservative or the like. Details for formulation and suitable pharmaceutically acceptable carriers may be found in “Remington's Pharmaceutical Sciences,” (19th ed., 1995), which is incorporated herein by reference.

Further, the pharmaceutical composition of the present invention is characterized in that it is administered via transmucosal routes. For example, administration routes for transmucosal delivery of the composition may include, but are not limited to, buccal, nasal, rectal, vaginal, urethral, throat, alimentary canal, peritoneal and ocular mucosae. Most preferably, the pharmaceutical composition of the present invention enables oral administration of the drug by delivery of the pharmacologically active substance via the alimentary canal mucosa.

A suitable dose of the pharmaceutical composition of the present invention may vary depending upon various factors such as formulation method, administration mode, age, weight and sex of patients, pathological conditions, diet, administration time, administration route, excretion rate and sensitivity to response. For oral administration, the composition is administered at a dose of preferably 0.001 to 100 mg/kg BW/day.

According to a method that can be easily practiced by a person having ordinary knowledge in the art to which the invention pertains, the pharmaceutical composition of the present invention may be formulated into a unit dosage form, or may be prepared in the form of a multi-dose form, using a pharmaceutically acceptable carrier and/or excipient. Here, the resulting formulation may be in the form of a solution, suspension or emulsion in oil or an aqueous medium, or otherwise may be in the form of an extract, a powder, a granule, a tablet or a capsule. The formulation may additionally comprise a dispersant or a stabilizer.

In the most preferred embodiment, the present invention provides a pharmaceutical composition for oral administration of insulin, comprising (a) a conjugate comprising a therapeutically effective amount of insulin covalently bound to chitosan, and (b) a pharmaceutically acceptable carrier.

The pharmaceutical composition for treatment of diabetes according to the present invention enables oral administration of insulin. Generally, diabetic patients are given an insulin injection. Such an administration method is very inconvenient to patients in several aspects. However, the pharmaceutical composition for treatment of diabetes according to the present invention may lead to remarkable improvement in diabetic treatment regimens due to the possibility of oral administration.

Upon comparing an in vivo blood glucose-lowering effect of the insulin-chitosan conjugate prepared according to the present invention with that of free insulin not bound to chitosan, it was confirmed through an experimental example of the present invention that the conjugate of the present invention exerts significantly higher blood glucose-lowering effects.

Further, it was also confirmed that the insulin-chitosan conjugate of the present invention exhibits an excellent absorption rate through a mucous membrane (particularly, the gastrointestinal mucosa).

In another most preferred embodiment, the pharmaceutical composition of the present invention provides a pharmaceutical composition for oral administration of paclitaxel, comprising (a) a conjugate comprising a therapeutically effective amount of paclitaxel covalently bound to chitosan, and (b) a pharmaceutically acceptable carrier.

The pharmaceutical composition comprising the paclitaxel-chitosan conjugate of the present invention exerts an excellent anti-cancer effects even by transmucosal administration, particularly oral transmucosal administration.

Upon comparing an in vivo anti-cancer effect of the paclitaxel-chitosan conjugate of the present invention with that of a free anti-cancer drug not bound to chitosan, it was confirmed through an experimental example of the present invention that the conjugate of the present invention exerts significantly higher anti-cancer effects.

Further, it was also confirmed that the paclitaxel-chitosan conjugate of the present invention exhibits an excellent absorption rate from a mucous membrane (particularly, gastrointestinal mucous membrane).

### Transmucosal Delivery of Pharmacologically Active Substances

In yet another aspect, the present invention provides a method for in vivo delivery of a pharmacologically active substance, via a transmucosal route, which comprises the steps of: (a) preparing a conjugate by binding covalently the pharmacologically active substance to a mucoadhesive polymer via a linker; and (b) administering the conjugate to a subject via the transmucosal route.

Preferably, the method of the present invention comprises (a-1) binding the pharmacologically active substance to the linker, and (a-2) conjugating the pharmacologically active substance of step (a-1) with the mucoadhesive polymer via the linker.

Preferably, the method of the present invention comprises (a-1) binding the pharmacologically active substance to the linker (a-2) binding the linker to the mucoadhesive polymer, and (a-3) conjugating the pharmacologically active substance of step (a-1) with the mucoadhesive polymer of step (a-2) via the linker.

### Prevention of Inhibition by P-glycoprotein and Increasing Bioavailability of Pharmacologically Active Substances

In further aspect of this invention, there is provided a method for increasing the transmucosal absorption of a pharmacologically active substance of which transmucoal absorption is inhibited by P-glycoprotein, which comprises the steps of: (a) preparing a conjugate by binding covalently the pharmacologically active substance to chitosan or its derivative via a linker; and (b) administering the conjugate to a subject via the transmucosal route.

A multitude of drugs, in particular, hydrophobic drugs do not show sufficient Bioavailability. Therefore, many researchers have made intensive researches to provide carrier systems and strategies that will enhance the bioavailability of such drugs in the gastrointestinal tract. One of strategies to enhance the bioavailability of hydrophobic drugs includes the utilization of P-glycoprotein (P-gp) inhibitors in formulations in an effort to increase absorption. Many drugs are substrates for the P-gp, which acts as an efflux pump. Exemplified P-gp inhibitors include cyclosporin A, poloxamers, polysorbates, verapamil and ketoconazole.

Interestingly the present inventors have found that the chitosan conjugate of this invention overcomes the shortcomings associated with the inhibition of the transmucosal absorption of drugs by P-glycoprotein, thereby dramatically increasing the bioavailability of various drugs.

According to a preferred embodiment, the pharmacologically active substance of which transmucoal absorption is inhibited by P-glycoprotein and is enhanced by the present conjugate system includes proteins; peptides; anti-cancer drugs such as cisplatin, methotrexate, paclitaxel, daunorubicin, doxorubicin, vincristine, vinblastine, carboplatin, procarbazine, mechlorethamine, cyclophosphamide, ifosfamide, melphalan, chlorambucil, bisulfan, docetaxel, camptothecin, nitrosourea, dactinomycin (actinomycin-D), bleomycin, plicomycin, mitomycin, etoposide, tamoxifen, transplatinum, 5-fluorouracil, adriamycin, quinolone, ciprofloxacin, progesterone, teniposide, estradiol, epirubicin and taxanes; prostaglandins; amphotericin B; vitamin E; steroids such as testosterone, beclomethasone, cortisone, dexamethasone, trimicinolone, aldosterone, methylprednisolone and betamethasone valerete; antiepileptic drugs such as phenytoin; antidepressant such as citacitalopram, thioperidone, trazodone, trimipramine, amitriptyline and phenothiazines; antipsychotic drugs such as fluphenazine, haloperidol, thioridazine, and trimipramine; protease inhibitors such as amprenavir, indinavir, lopinavir, nelfinavir, ritonavir and saquinavir; calcium blockers such as bepridil, diltiazem, flunarizine, lomerizine, secoverine, tamolarizine, verapamil, nicardipine, prenylamine and fendiline; and cardiac drugs such as digoxin, diltiazem, verapamil and talinolol.

Among the various accomplishments and advantages are the following:

(i) The conjugate of the present invention exhibits an excellent absorption rate in biological mucous membranes, particularly mucous membranes of the alimentary canal (especially the gastrointestinal tract).

(ii) Because the mucoadhesive polymer used as the carrier of a target drug is highly biocompatible and biodegradable in vivo, the conjugate of the present invention is safe and also exhibit excellent safety even with chrome administration.

(iii) Consequently, the pharmaceutical composition of the present invention exhibits superior bioavailability even upon oral administration, thus making it possible to achieve treatment of diseases via oral administration.

(iv) Oral administration of the pharmaceutical composition of the present invention leads to significant improvements in medication compliance of the patients, as compared to conventional injection medications.

Aspects of the present invention are described in further detail in the examples set forth below, which are intended to be more concretely illustrative; noting, however, that the scope of the present invention as set forth in the appended claims is not limited to or by the following examples.

### EXAMPLES

### I. Insulin-Chitosan Conjugates

### Example 1

### Preparation of Insulin Intermediate Having Insulin Bound to Linker

0.1 g (17.22×10⁻⁶mol) of insulin (Serologicals Corp.) was dissolved in 10 mL of a hydrochloric acid solution, and 0.008 g (25.83×10⁻⁶ mol) of N-succinimidyl 3-(2-pyridyldithio) propionate (SPDP, Pierce) was dissolved in 0.2×10⁻³ mL of DMF (Sigma) which was then added to the insulin solution. In order to achieve regioselective conjugation of SPDP with the 29th amino acid lysine on the B chain (B29) of an insulin molecule, the aforementioned mixed solution was adjusted to a range of pH 9 to 10 using aqueous NaOH and stirred at room temperature for 30 min. The resulting stirred solution was subjected to reverse-phase HPLC (Shimadzu) separation and freeze-drying (lyophilization) to thereby prepare an insulin intermediate product (see Reaction Scheme 1).

### Example 2

### Preparation of Chitosan Intermediate Having Chitosan Bound to Linker

Each 0.1 g (16.67×10⁻⁶ mol), moles of monomer=0.67×10⁻³ mol) of chitosan with a different molecular weight of 3000, 6000 and 9000 (KITTOLIFE, Co., Ltd., Seoul, Korea) was dissolved in 2 mL of a phosphate buffer solution (PBS), and 0.016 g (50.01×10⁻⁶ mol) of SPDP was dissolved in 0.2×10⁻³ mL of DMF which was then added to the aforementioned chitosan solution, followed by stirring at room temperature for 2 hours. Acetone was added to the resulting stirred solution to thereby precipitate pellets. The resulting pellets were dissolved in distilled water and freeze-dried to thereby prepare a chitosan intermediate product (see Reaction Scheme 1).

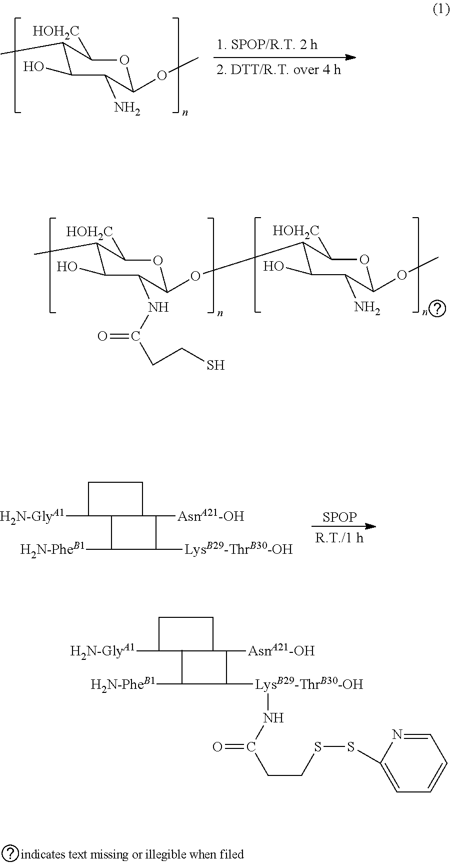

### Example 3

### Construction of Insulin-Chitosan Conjugate

In order to reduce the chitosan intermediate, 0.008 g (1.24×10⁻⁶ mol) of the chitosan intermediate prepared in Example 2 and 0.3 mL of DTT (24.9×10⁻⁶ mol) (Pierce) were dissolved in 0.3 mL of PBS and stirred at room temperature for 4 hours, 0.005 g (0.83×10⁻⁶ mol) of the insulin intermediate prepared in Example 1 was dissolved in a citrate buffer solution (500 μl), the reduced chitosan intermediate solution (100 μl ) was added thereto, and the resulting mixture was stirred at room temperature for 12 to 24 hours. The stirred mixture was subjected to reverse-phase HPLC separation and freeze-drying to thereby prepare an insulin-chitosan conjugate (see Reaction Scheme 2).

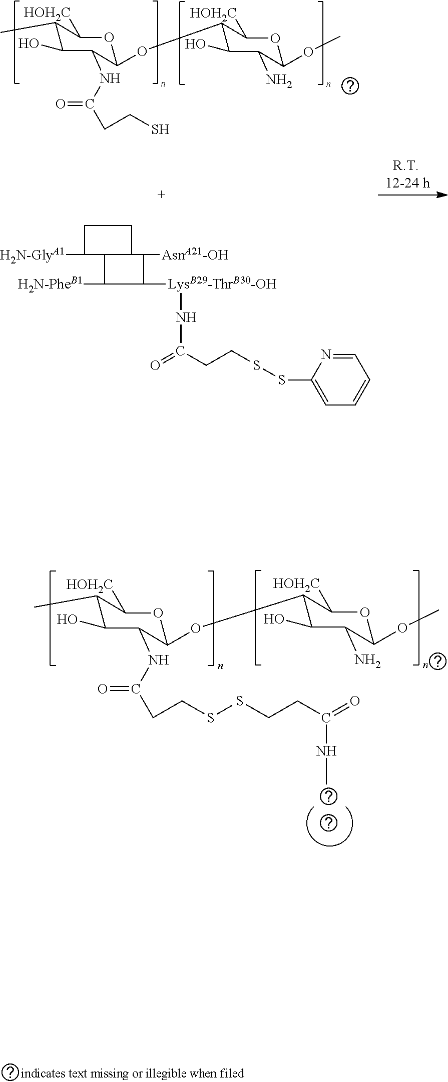

### Experimental Example 1

### Determination of Substitution Degree with Linker in chitosan intermediate

¹H NMR analysis was tamed out to determine the SPDP-substituted degree in the chitosan intermediate of the present invention. The substitution degree of the linker was calculated in a D₂O solvent by integral calculus. The number of substituted molecules thus measured is given in Table 1 below.

TABLE 1

| Chitosan MW | Number of substituted molecules |
|---|---|
| 3000 | 3 |
| 6000 | 1.9 |
| 9000 | 1.6 |

As shown in Table 1, it can be seen that low-molecular weight chitosan is more easily substituted with the linker since the substitution degree of the linker decreases in proportion to an increase in the molecular weight of chitosan.

### Experimental Example 2

### Determination of Insulin Content in Insulin-Chitosan Conjugate

In order to determine an amount of insulin contained in an insulin-chitosan conjugate of the present invention (a conjugate using chitosan of MW 6000). 1 mg of the insulin-chitosan conjugate was dissolved in 1 mL of a citrate buffer solution and an absorbance was measured at a wavelength of UV 275 nm. The standard curve was plotted by dissolving insulin (0.1, 0.5, 1 and 2 mg) in 1 mL of a citrate buffer solution and measuring the absorbance at the given wavelength. Using the thus-obtained standard curve, the amount of insulin contained in the insulin-chitosan conjugate was calculated. As a result, the content of insulin in the conjugate was 44%.

### Experimental Example 3

### Determination of in Vivo Insulin Activity Using Insulin-Chitosan Conjugate

An insulin-chitosan conjugate of the present invention (a conjugate using chitosan of MW 6000) was dissolved in a citrate buffer solution and then diluted with physiological saline to prepare an insulin-chitosan conjugate solution at an insulin concentration of 1 U/mL. Diabetes-induced male Wistar rats (6 to 7-weeks old) were fasted for 6 hours prior to administration of insulin, and blood was collected from the tail veins of the animals and the blood glucose level was determined. The thus-obtained value was used as an initial value. Immediately after determination of the blood glucose level, a 0.5 IU/kg insulin- or 1 IU/kg insulin-chitosan conjugate (Insulin-6K LMWC) was intravenously injected to the tail veins of the animals. 0.5 IU is equivalent to 17.4 μg of insulin. In addition, animals were given subcutaneous (s.c.) injection of 0.5 IU/kg insulin (control).

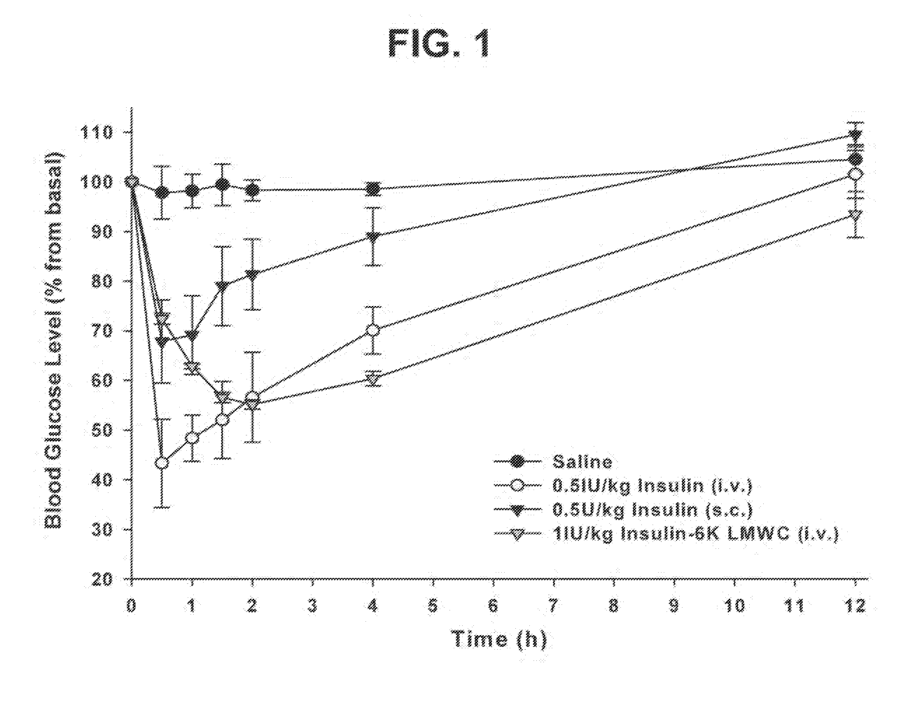

As shown in FIG. 1, a physiological activity of insulin contained in the insulin-chitosan conjugate solution of the present invention (-∇-) exhibited about 40% of the insulin solution control, thus confirming that the conjugate of the present invention has a normal physiological activity.

### Experimental Example 4

### In Vivo Oral Administration Studies of Insulin-Chitosan Conjugate

An insulin-chitosan conjugate (a conjugate using chitosan of MW 3000, 6000 or 9000 Da) was dissolved in a citrate buffer solution and then diluted with physiological saline to prepare an insulin-chitosan conjugate solution at an insulin concentration of 100 U/mL. Diabetes-induced rats were tasted for 6 hours, and blood was collected from the tail veins of animals and the blood glucose level was determined. The thus-obtained value was used as an initial value poor to administration of the drug. The experimental animals were given oral administration of the above-prepared insulin-chitosan conjugate solution at a dose of 50 IU/kg using a gastric sonde (50 IU is equivalent to 1.77 mg of insulin). As a control, animals were given oral administration of 50 IU/kg insulin and chitosan of MW 9000 Da in the same mariner as above. On time points of 1, 2, 3, and 4 hours after administration of the drug, blood was collected from the tail veins of animals and the blood glucose level was determined. The blood glucose level at each time point was calculated by taking the initial value prior to administration of the drug to be 100%.

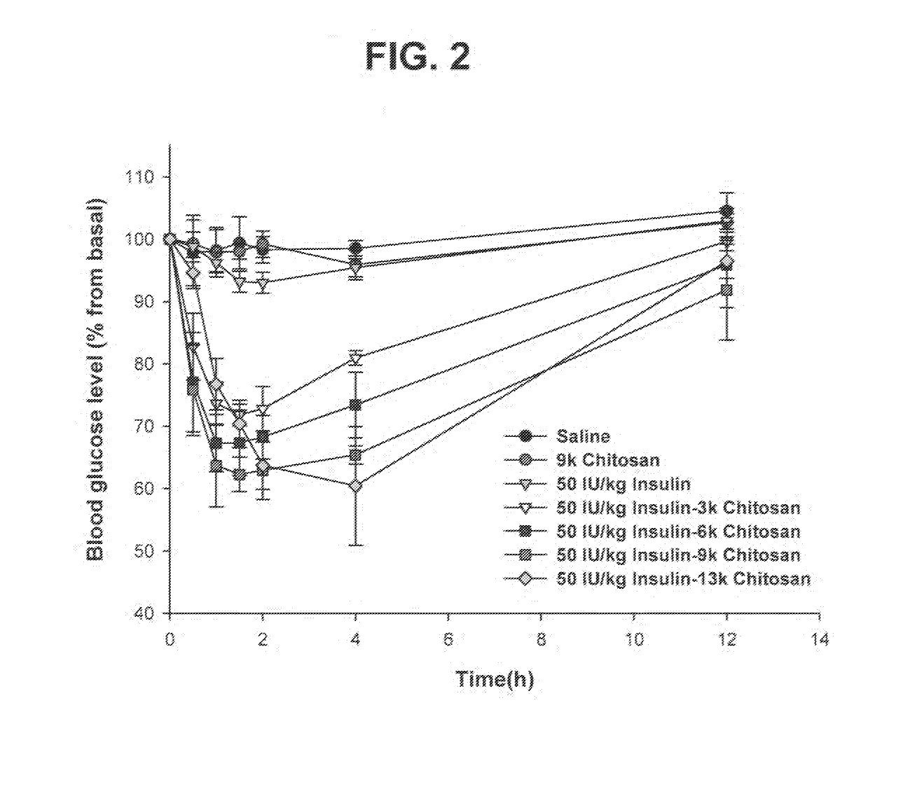

As shown in FIG. 2, an experimental group of rat with administration of the insulin-chitosan conjugate solution of the present invention at a dose of 50 IU insulin/kg exhibited more than a 40% decrease in the blood glucose level 2 hours later, as compared to the initial blood glucose level. Whereas, animal groups with oral administration of insulin-free saline, insulin itself and chitosan itself exhibited no lowering of the blood glucose levels.

Then, the bioavailability of conjugates were calculated from the degree of blood glucose control (area under curve, AUC) obtained in FIG. 2 after oral administration of each insulin-chitosan conjugate. The results thus obtained are summarized in Table 2 below. Analysis was conducted by administering insulin to homologous rats via IV and SC injection and taking the degree of blood glucose control thus obtained to be 200% bioavailability. In addition, as a control, known bioavailability of insulin, a protease-chitosan conjugate and a thiolated chitosan-insulin tablet preparation containing glutathione, (a reducing agent) (Krauland A H, et al., J. Control Release, 24; 95(3): 547-555 (2004)) was compared.

TABLE 2

| | Insulin-3K | Insulin-6K | Insulin-9K | |
|---|---|---|---|---|
| Items | LMWC | LMWC | LMWC | Others* |
| Insulin admini- | 1.77 | 1.77 | 1.77 | 11 |
| stered (mg/kg) | | | | |
| Protease inhib- | Not added | Not added | Not added | Added |
| itor | | | | |
| Bioavailability | 0.55 ± 0.11 | 0.77 ± 0.16 | 1.0 ± 0.13 | 0.65 ± 0.16 |
| (%) (IV injec- | | | | |
| tion: 100%) | | | | |
| Bioavailability | 1.89 ± 0.32 | 2.66 ± 0.38 | 1.69 ± 0.29 | 1.69 ± 0.42 |
| (%) (SC injec- | | | | |
| tions: 100%) | | | | |

*Group with administration of a thiolated chitosan-insulin tablet

As indicated in Table 2, the insulin-chitosan conjugate of the present invention also exhibited excellent bioavailability.

### II. Calcitonin-Chitosan Conjugates

### Example 4

### Preparation of Calcitonin Intermediate Having Calcitonin Bound to Linker

0.059 g (17.22×10⁻⁶ mol) of salmon calcitonin (Serologicals Corp.) was dissolved in 10 mL of a borate buffer solution (pH 8-9), and 0.008 g (25.83×10⁻⁶ mol) of N-succinimidyl 3-(2-pyridyldithio) propionate (SPDP, Pierce) was dissolved in 0.2×10⁻³ mL of DMF (Sigma), which was then added to the calcitonin solution. In order to achieve regioselective conjugation of SPDP with the 32^{th} amino acid proline on the N-terminal portion of calcitonin, the aforementioned mixed solution was adjusted to a range of pH 8 to 9 using aqueous NaOH and stirred at room temperature for 1 hr. The resulting stirred solution was subjected to reverse-phase HPLC (Shimadzu) separation and freeze-drying (lyophilization) to thereby prepare an salmon calcitonin intermediate product (see Scheme 3).

### Example 5

### Preparation of Chitosan Intermediate Having Chitosan Bound to Linker

0.1 g (16.67×10⁻⁶mol, moles of monomer=0.67×10⁻³ mol) of chitosan with a molecular weight of 6000 (KITTOLIFE, CO., Ltd., Seoul, Korea) was dissolved in 2 mL of PBS, and 0.016 g (50.01×10⁻⁶ mol) of SPDP was dissolved in 0.2×10⁻³ mL of DMF, which was then added to the aforementioned chitosan solution, followed by stirring at room temperature for 2 hours. Acetone was added to the resulting stirred solution to thereby precipitate pellets. The resulting pellets were dissolved in distilled water and freeze-dried to thereby prepare a chitosan intermediate product (See Scheme 4).

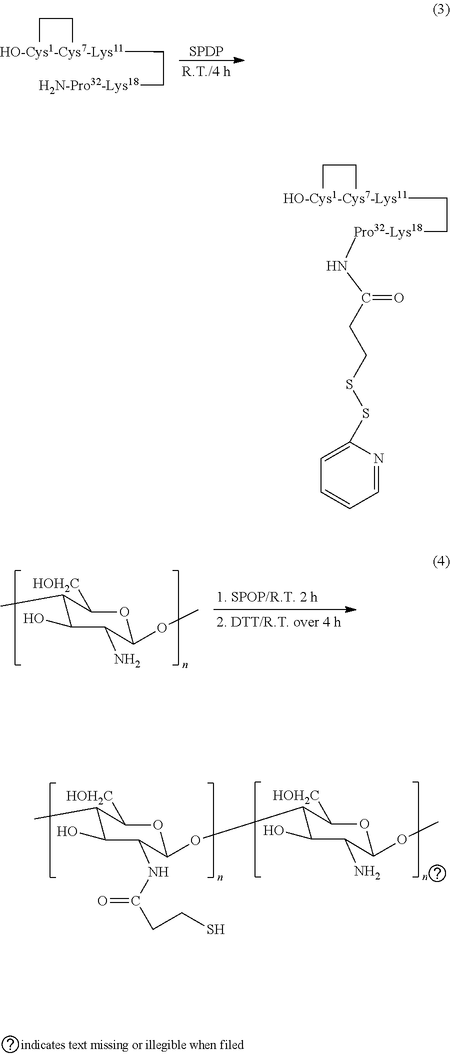

### Example 6

### Construction of Calcitonin-Chitosan Conjugate

In order to reduce the chitosan intermediate, 0.008 g (1.24×10⁻⁶ mol) of the chitosan intermediate prepared and 0.3 mL of DTT (24.9×10⁻⁶ mol) were dissolved in 0.3 mL of PBS and stirred at room temperature for 4 hours, 0.005 g (0.83×10⁻⁶ mol) of the calcitonin intermediate prepared was dissolved in a citrate buffer solution (500 μl) the reduced chitosan intermediate solution (100 μl) was added thereto, and the resulting mixture was stirred at room temperature for 12 to 24 hours. The stirred mixture was subjected to reverse-phase HPLC separation and freeze-drying to thereby prepare an calcitonin-chitosan conjugate (see Scheme 5).

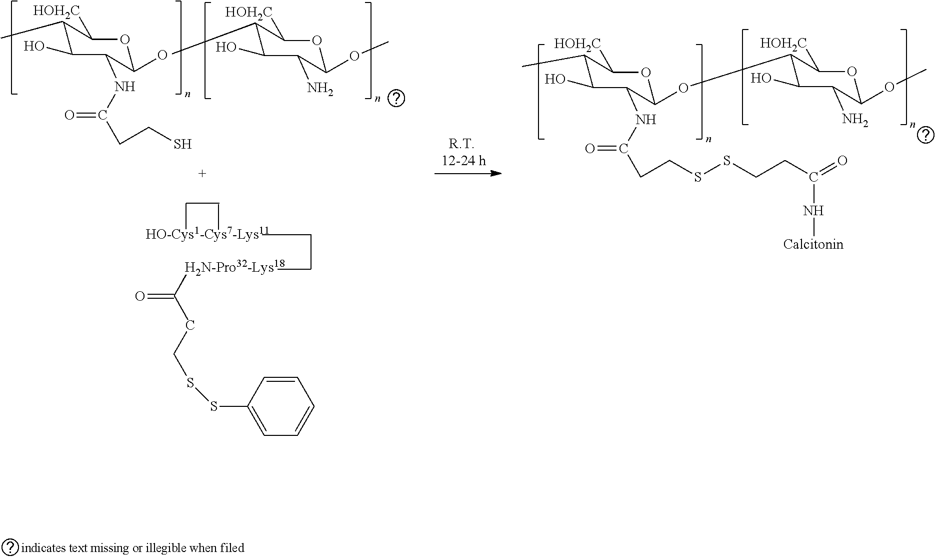

### Experimental Example 5

### Determination of Calcitonin Content in Calcitonin-Chitosan Conjugate

In order to determine an amount of calcitonin present in the calcitonin-chitosan conjugate prepared above, 1 mg of the calcitonin-chitosan conjugate obtained was dissolved in 1 mL of a phosphate buffer and an absorbance was measured at a wavelength of UV 2.75 nm. The standard curve was plotted by dissolving salmon calcitonin (0.1, 0.5, 1.0 and 2.0 mg) in 1 mL of phosphate buffer and measuring the absorbance at the given wavelength. Using the thus-obtained standard curve, the amount of calcitonin contained in the calcitonin-chitosan conjugate was calculated. As a result, file content of calcitonin in the conjugate was determined 32% for chitosan of MW 6000.

### Experimental Example 6

### Determination of Calcitonin Activity in Calcitonin-Chitosan Conjugate

The calcitonin-chitosan conjugate of the present invention (a conjugate using chitosan of MW 6000) was dissolved in a phosphate buffer solution and then diluted with physiological saline to prepare a calcitonin-chitosan conjugate solution at a calcitonin concentration of 10⁻¹²-10⁻⁷ M. T-47D cells (human breast cancer cell line, ATCC) were plated into 96-well plates at a density of 1.5×10⁴ cells/well and then cultured for 24 hr, after which they were cultured for 10 min in HBSS medium (Gibco) supplemented with 0.1% BSA (Gibco) and 1 mM IBMX (Sigma). The cultured cells were incubated with the salmon calcitonin solution for 1 hr. The level of cAMP produced by calcitonin was measured using cAMP Enzymeimmuno assay kit (Amersham, Uppsala, Sweden). As a control, a salmon calcitonin not conjugated with chitosan was used.

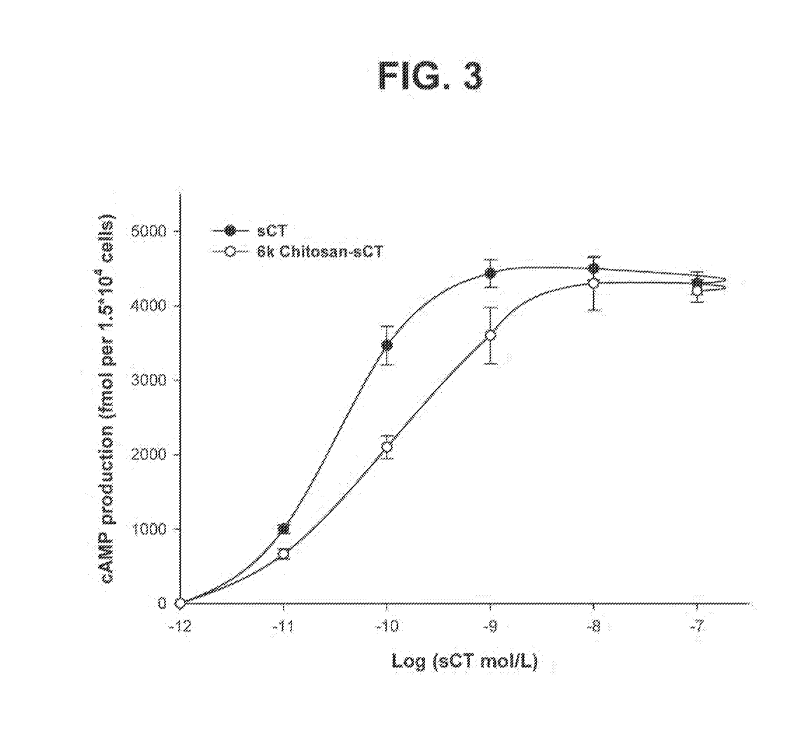

As shown in FIG. 3, the activity of calcitonin contained in the calcitonin-chitosan conjugate solution of the present invention was measured to be about 46% of the calcitonin solution control, verifying that the conjugate of the present invention has a normal physiological activity.

### Experimental Example 7

### Oral Administration Studies on Calcitonin-Chitosan Conjugates

The calcitionin-chitosan conjugate (a conjugate, using chitosan of MW 6000 Da) was dissolved in a phosphate buffer and then diluted with physiological saline to prepare a calcitonin-chitosan conjugate solution at a calcitonin concentration of 100 μg/mL. Rats were fasted for 6 hours and given oral administration of the above-prepared calcitonin-chitosan conjugate solution at a dose of 100 μg/kg using a gastric sonde. As a control, rats were given oral administration of 100 μg/kg calcitonin in the same manner as above. At time points of 1, 3, 6 and 12 hours after administration of the drug, blood was collected from the tail veins of rats and the calcitonin levels in plasma were determined.

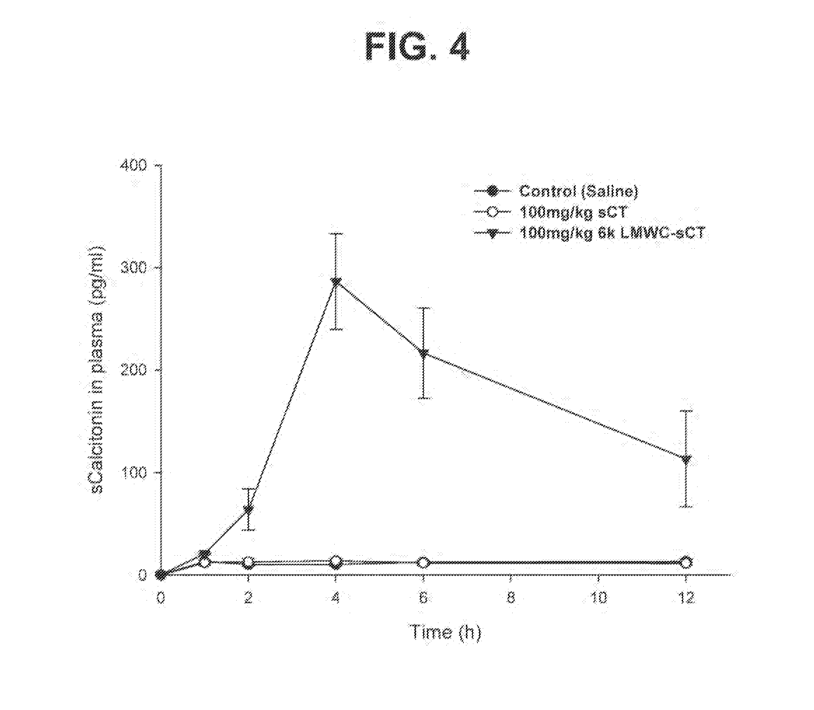

As represented in FIG. 4, the salmon calcitonin-chitosan conjugate of this invention shows higher level in blood than bare calcitonin and highest blood level at 4 hour post-administration.

### III. Paclitaxel-Chitosan Conjugates

### Example 7

### Preparation of Paclitaxel Intermediate Having Paclitaxel Bound to Linker

0.1 g (0.117×10⁻³ mol) of paclitaxel (Samyang Genex Corp., Daejeon, Korea) was dissolved in 5 mL of a dichloromethane solution, and 0.015 g (0.152×10⁻³ mol) of succinic anhydride (Sigma, St. Louis, Mo.) and 12.9×10⁻³ mL (0.160×10⁻³ mol) of pyridine (Sigma) were added to the paclitaxel solution. The resulting mixture was stirred at room temperature for 3 days. The resulting stirred solution was purified by silica column chromatography and dried to prepare a paclitaxel/succinic acid derivative.

### Example 8

### Construction of Paclitaxel-Chitosan Conjugate

0.1 g (0.105×10⁻³ mol) of a paclitaxel/succinic acid derivative, 1-ethyl-3-(3-dimethylaminopropyl)carbodiimide hydrochloride (EDC) (Sigma) and N-hydroxysuccinimide (NHS) (Sigma) were dissolved in 3 mL of DMF, and the resulting mixture was stirred at room temperature for 4 hours (see Scheme 6). 0.2 g (66.67×10⁻⁶ mol) of chitosan of MW 3000 and 6000 (KITTOLIFE, Co., Ltd., Seoul, Korea) was dissolved in a borate buffer solution (3 mL) and DMF (9 mL), which was then added to the above stirred solution and stirred at room temperature for 4 hours (see Scheme 6). The reaction solution was dialyzed against distilled water and freeze-dried to thereby obtain a paclitaxel-chitosan conjugate.

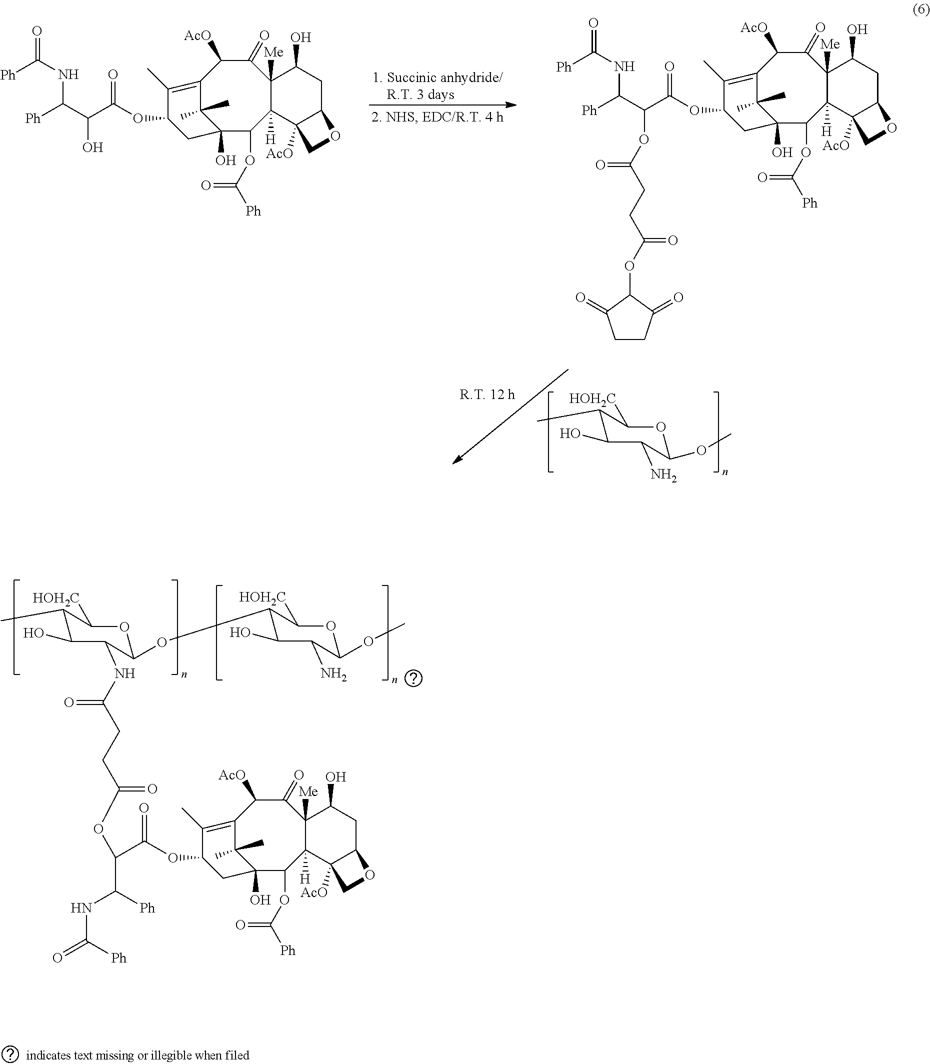

### Experimental Example 8

### Determination of Paclitaxel Content in Paclitaxel-Chitosan Conjugate

In order to determine an amount of paclitaxel contained in a paclitaxel-chitosan conjugate of the present invention, 0.1 mg of the paclitaxel-chitosan conjugate obtained in Example 8 was dissolved in 1 mL of acetonitrile/water and an absorbance was measured at a wavelength of UV 227 nm. The standard curve was plotted by dissolving paclitaxel (5, 10, 12.5, 20 and 25 mg) in 1 mL of acetonitrile/water and measuring the absorbance at the given wavelength. Using the thus-obtained standard curve, the amount of paclitaxel contained in the paclitaxel-chitosan conjugate was calculated. As a result, the content of paclitaxel in the conjugate was 15-20% and 10-15% for chitosan of MW 3000 and 6000, respectively.

### Experimental Example 9

### In Vitro Cytotoxicity Test Using Paclitaxel-Chitosan Conjugate

A paclitaxel-chitosan conjugate of the present invention (3000 and 6000 Da) was dissolved in dimethyl sulfoxide (DMSO) and diluted with a cell culture medium to prepare paclitaxel-chitosan conjugate solutions at a paclitaxel concentration of 0.01, 0.05, 0.1, 0.25, 0.5 and 1 μg/mL. B16F10 murine melanoma cells and MDA-MB-231 human breast carcinoma (KTCC) were cultured in a 96-well plate at a cell density of 5×10³ cells/well for 24 hours and were treated with the above-prepared paclitaxel solution fur 48 hours. Thereafter the cell viability was measured using an MTT cell viability kit (Molecular Probe, Netherlands). 50 μl of MTT was added to cells which were then cultured at 37° C. for 4 hours. Then, the supernatants were completely eliminated and 100-well of DMSO was added to the 96-well plate. The absorbance was measured using a microplate reader. The cell viability was calculated according to the following Equation (1):

Cell viability (%)=(OD₅₇₀(Sample)/OD₅₇₀(Control))×100 (Equation 1)

A non-conjugated paclitaxel solution was used as a control.

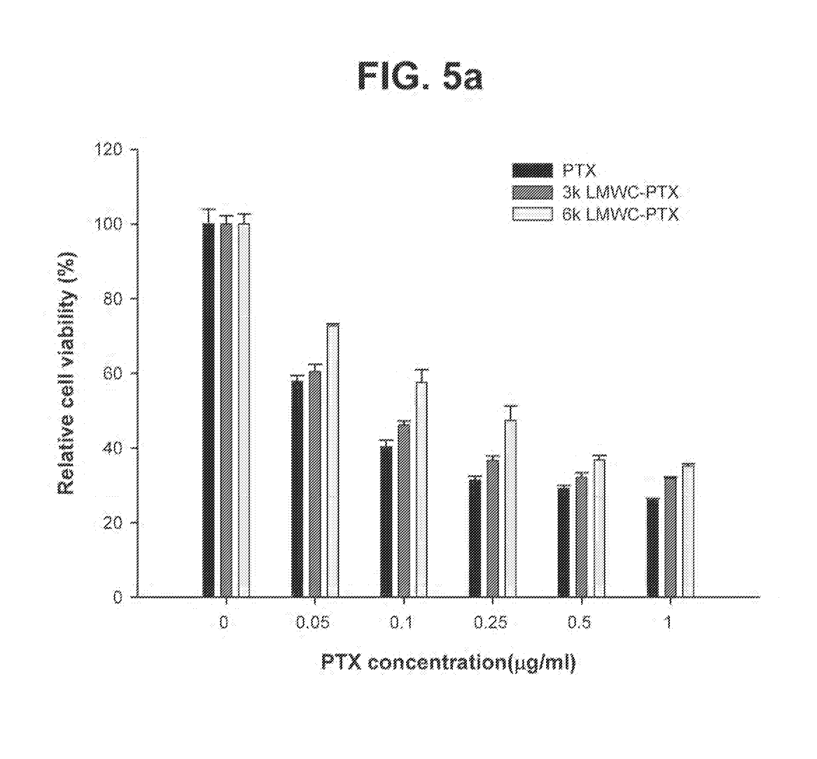

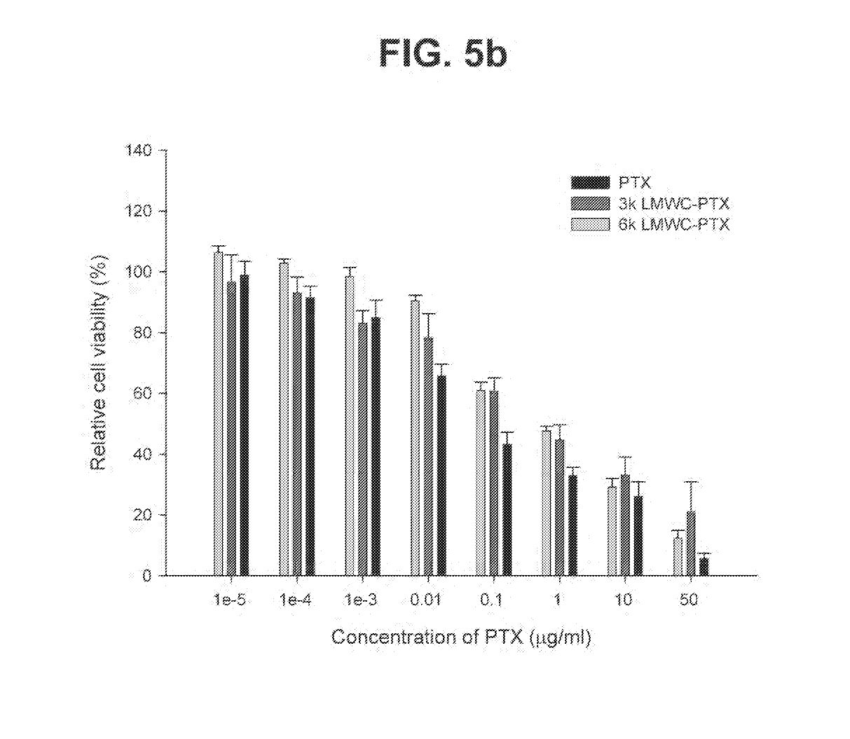

As shown in FIGS. 5a and 5b, it was confirmed that the cytotoxicity of paclitaxel contained in the paclitaxel-chitosan conjugate solution of the present invention was similar to that of the non-conjugated paclitaxel.

### Experimental Example 10

### Effects of P-Glycoprotein (P-gp) Inhibitor After Oral Administration of Paclitaxel-Chitosan Conjugate in Vivo

ICR mice (male, 25-30 g) were fasted for 12 hr before dosing. Mice were anesthetized with diethyl ether and administered with a single oral dose of paclitaxel or paclitaxel-chitosan conjugates with or without P-gp inhibitor (cyclosporine A, Sigma, 15 mg/kg) through an oral gavage that was carefully passed down the esophagus into the stomach. The drug solutions were prepared in 10% DMSO solution. The total volume of the administered drug solution was 0.2 ml. Blood (450 μl) was collected from a capillary in the retroorbital plexus and directly mixed with 50 Al of sodium citrate (3.8% solution); the sample was then immediately centrifuged at 3000 rpm at 4° C. for 20 min. The concentrations of paclitaxel in plasma were measured using HPLC after extraction.

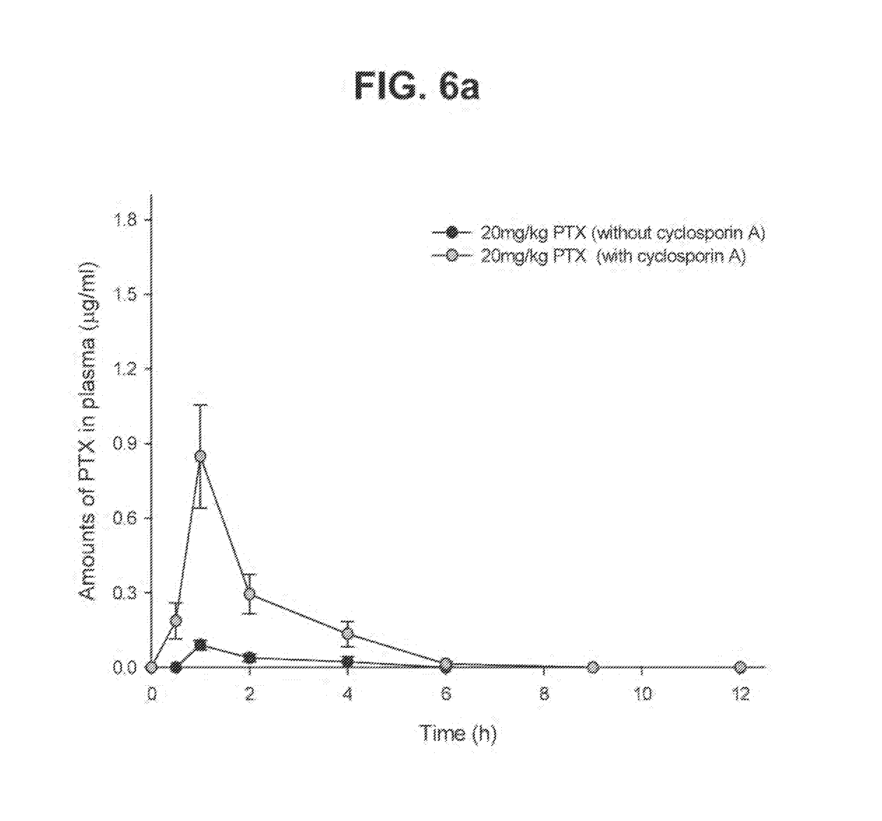

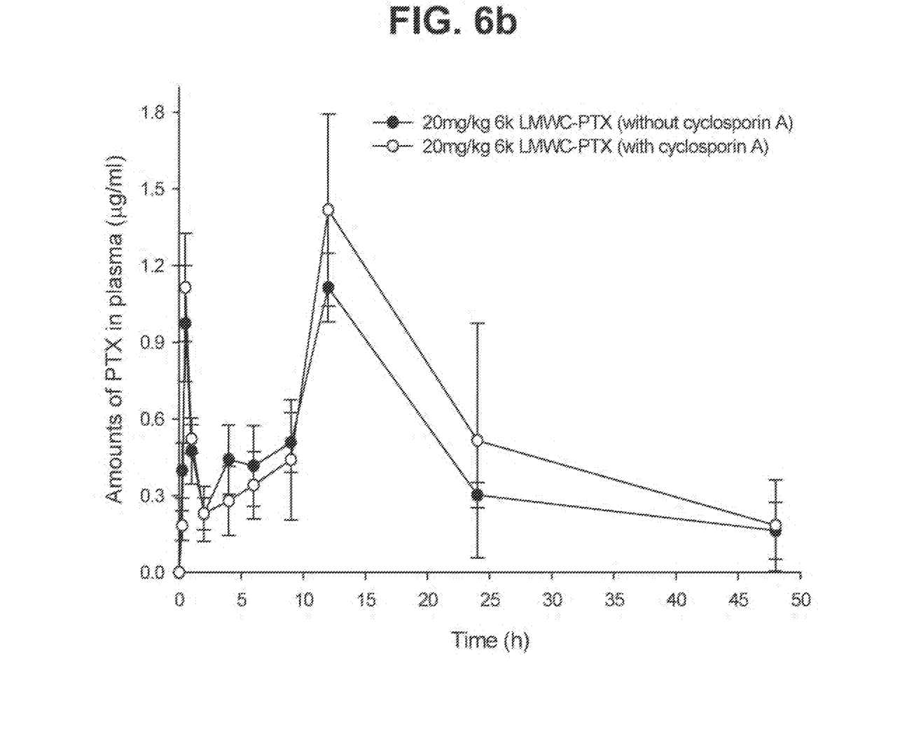

As shown in FIG. 6a, paclitaxel is very poorly absorbed after oral administration with maximum plasma concentration (Cₘₐₓ) of 0.09±0.02 μg/ml. Coadministration of cyclosporine A with paclitaxel resulted in a significant increase in plasma concentration of paclitaxel. The maximum plasma concentration (Cₘₐₓ) was 9.3-fold higher, when coadministrated with cyclosporine A. However, paclitaxel-chitosan conjugate is absorbed after oral administration with maximum plasma concentration (Cₘₐₓ) of 0.97±0.23 μg/ml. Also, the maximum plasma concentration (Cₘₐₓ) of paclitaxel did not increase after coadministration with cyclosporine A (FIG. 6b)

### Experimental Example 11

### Inhibitory Effects of Oral Administration of Paclitaxel-Chitosan Conjugate on Tumor

B16F10 melanoma cells were subcutaneously transplanted at a cell density of 5×10⁶ cells/mice into a dorsal region of C57BL6 male mice (mean body weight: 25 g). When the tumor mass has reached a desired size of about 50 to 100 mm³, animals were divided into a treatment group and a control group. Experiments were carried out for mouse groups, each consisting of 5 to 6 animals having the tumor, simultaneously with observation of changes. Animals were given oral administration of the drug or physiological saline for about 30 days, starting on day 10 after tumor transplantation. Paclitaxel and the paclitaxel-chitosan conjugate were administered to animals at a dose of 25 mg/kg for 5 days, with no administration for following two days. The control group was administered physiological saline, paclitaxel and chitosan. In order to confirm the degree of tumor growth, the size of tumor was daily measured using a calibrator. The tumor size was calculated according to the following Equation (2):

Tumor volume (mm³)=(Length×Width²)/2 (Equation 2).

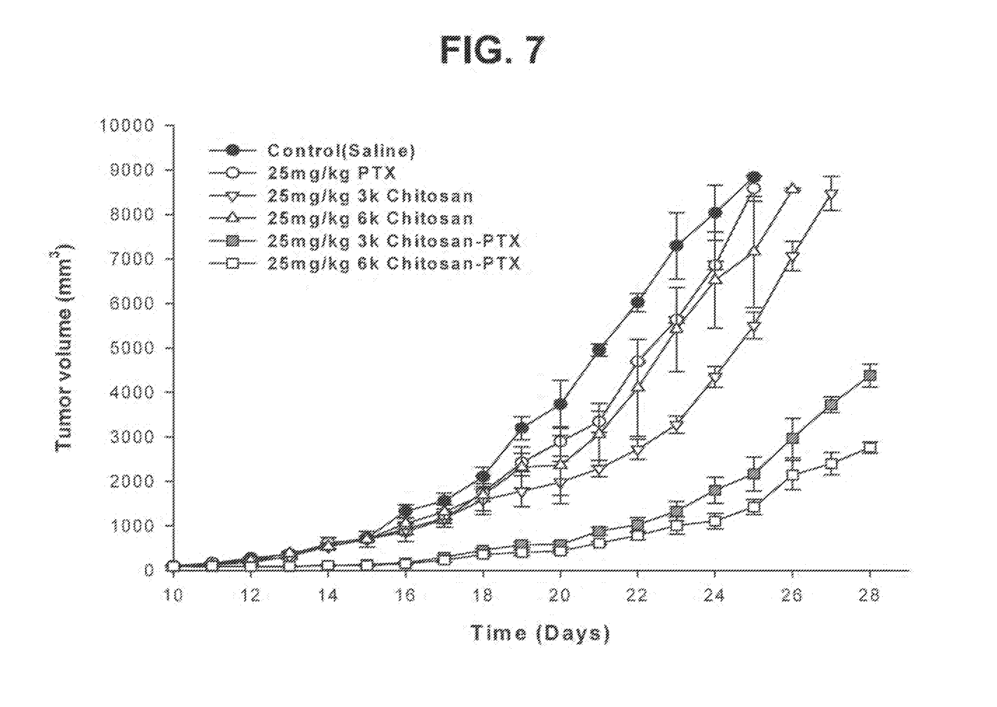

FIG. 7 is a graph showing an anti-cancer activity in mice with administration of paclitaxel and the paclitaxel-chitosan conjugate, respectively. the paclitaxel-administered group exhibited no significant difference in the tumor size, as compared to that of the control group. However, it can be seen that the group with the administration of the paclitaxel-chitosan conjugate of the present invention exhibited a significant decrease in the tumor size, as compared to the control group.

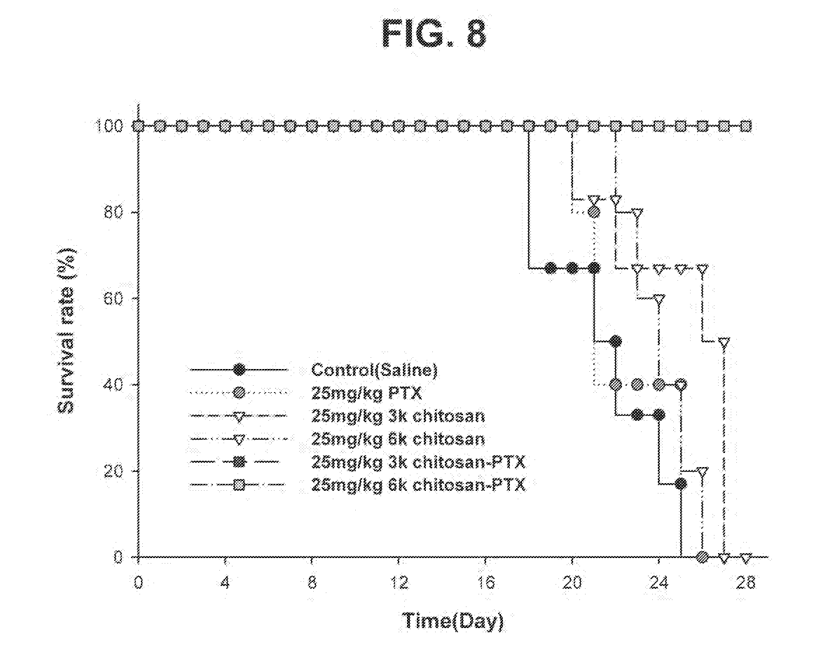

The survival rate of mice was also monitored simultaneously with measurement of the tumor size. When the tumor mass reached a size of more than 8000 mm³, the animals were euthanized. As shown in FIG. 8, the mice of the group with the administration of the paclitaxel-chitosan conjugate of the present invention exhibited a 100% survival rate for about 30 days, whereas the mice of the control group exhibited a 0% survival rate prior to 30 days.

### IV. Docetaxel-Chitosan Conjugates

### Example 9

### Preparation of Docetaxel Intermediate Having Docotaxel Bound to Linker

0.1 g (0.116×10⁻³ mol) of docetaxel (APIN, Oxon, UK) was dissolved in 5 mL of a dichloromethane solution, and 0.015 g (0.152×10⁻³ mol) of succinic anhydride (Sigma, St. Louis, Mo.) and 12.9×10⁻³ mL (0.160×10⁻³ mol) of pyridine (Sigma) were added to the docetaxel solution. The resulting mixture was stirred at room temperature for 3 days (see Scheme 4). The resulting stirred solution was purified by silica column chromatography and dried to give a docetaxel/succinic acid derivative.

### Example 10

### Construction of Docetaxel-Chitosan Conjugate

0.1 g (0.105×10⁻³ mol) of the docetaxel/succinic acid derivative, 1-ethyl-3-(3-dimethylaminopropyl)carbodiimide hydrochloride (EDC) (Sigma) and N-hydroxysuccunimide (NHS) (Sigma) were dissolved in 3 mL of DMF, and the resulting mixture was stirred at room temperature for 4 hours (see Scheme 7). 0.2 g (66.67×10⁻⁶ mol) of chitosan of MW 6000 (KITTOLIFE, Co., Ltd., Seoul, Korea) was dissolved in a borate buffer solution (3 mL) and DMF (9 mL), which was then added to the above stirred solution and stirred at room temperature for 4 hours (see Scheme 7). The reaction solution was dialyzed against distilled water and freeze-dried to thereby obtain a docetaxel-chitosan conjugate.

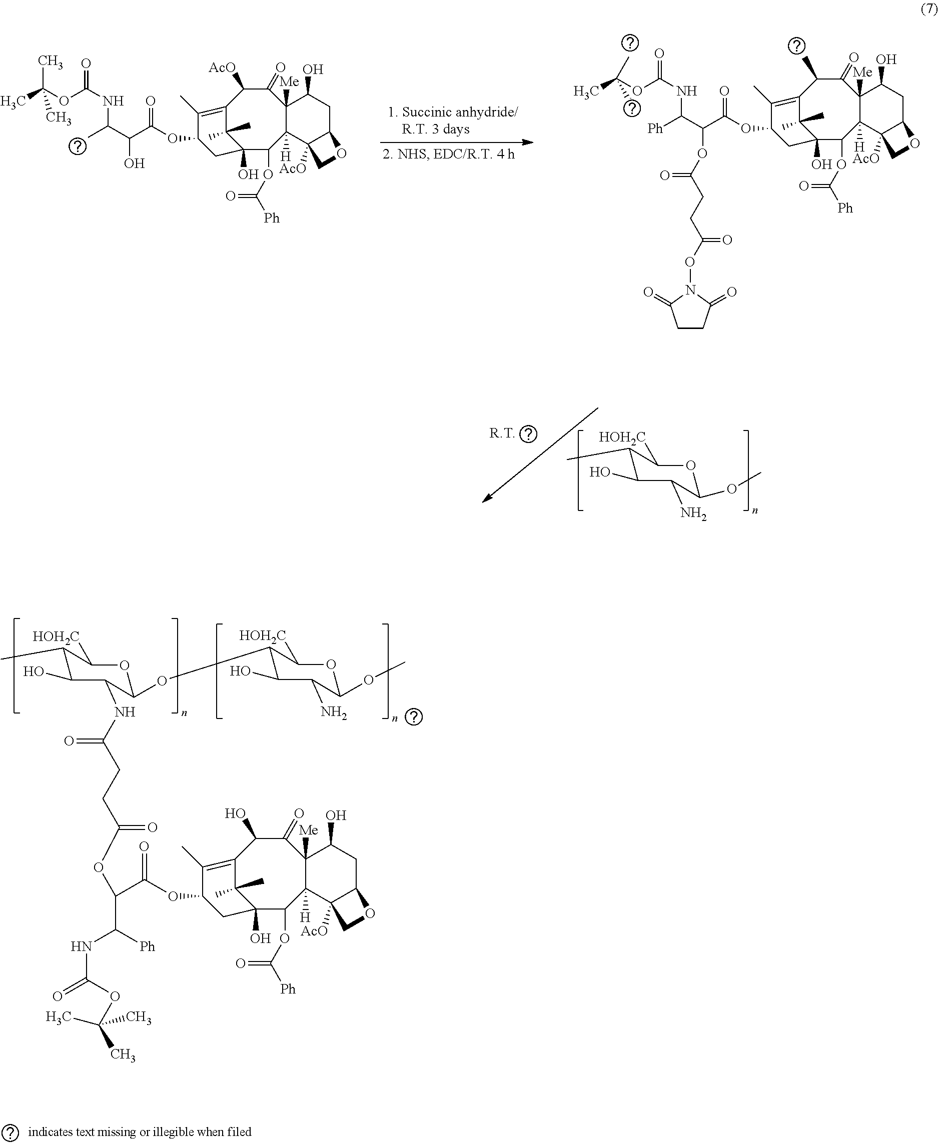

### Experimental Example 12

### Determination of Docetaxel Content in Docetaxel-Chitosan Conjugate

In order to determine an amount of docetaxel present in the docetaxel-chitosan conjugate prepared above, 0.1 mg of the docetaxel-chitosan conjugate obtained in Example 10 was dissolved in 1 mL of acetonitrile/water and an absorbance was measured at a wavelength of UV 227 nm. The standard curve was plotted by dissolving docetaxel (5, 10, 12.5, 20 and 25 μg) in 1 mL of acetonitrile/water and measuring the absorbance at the given wavelength. Using the thus-obtained standard curve, the amount of docetaxel contained in the docetaxel-chitosan conjugate was calculated. As a result, the content, of docetaxel in the conjugate was determined 15-20% for chitosan of MW 6000 .

### V. Doxorubicin-Chitosan Conjugates

### Example 11

### Preparation of Doxorubicin Intermediate Having Doxorubicin Bound to Linker

0.04 g (0.117×10⁻³ mol) of doxorubicin/HCl (HCl salt form, Boryung Pharmaceutical Co., Ltd, Seoul, Korea) was dissolved in 5 mL of anhydrous DMSO solution, and 0.015 g (0.15×10⁻³ mol) of succinic anhydride was added to the doxorubicin solution. The resulting mixture was stirred at room temperature for 3 days under dark conditions (see Scheme 8). The resulting stirred solution was purified by silica column chromatography and dried to give a doxorubicin/succinic acid derivative.

### Example 12

### Construction of Doxorubicin-Chitosan Conjugate

0.047 g (0.105×10⁻³ mol) of the doxorubicin/succinic acid derivative, 1-ethyl-3-(3-dimethylaminopropyl)carbodiimide hydrochloride (EDC) (Sigma) and N-hydroxysuccinimide (NHS) (Sigma) were dissolved in 3 mL of DMF, and the resulting mixture was stirred at room temperature for 4 hours (see Scheme 5). 0.2 g (66.67×10⁻⁶ mol) of chitosan of MW 6000 (KITTOLIFE, Co., Ltd., Seoul, Korea) was dissolved in a borate buffer solution (3 mL) and DMF (9 mL), which was then added to the above stirred solution and stirred at room temperature for 4 hours (see Scheme 8). The reaction solution was dialyzed against distilled water and freeze-dried to thereby obtain a doxorubicin-chitosan conjugate.

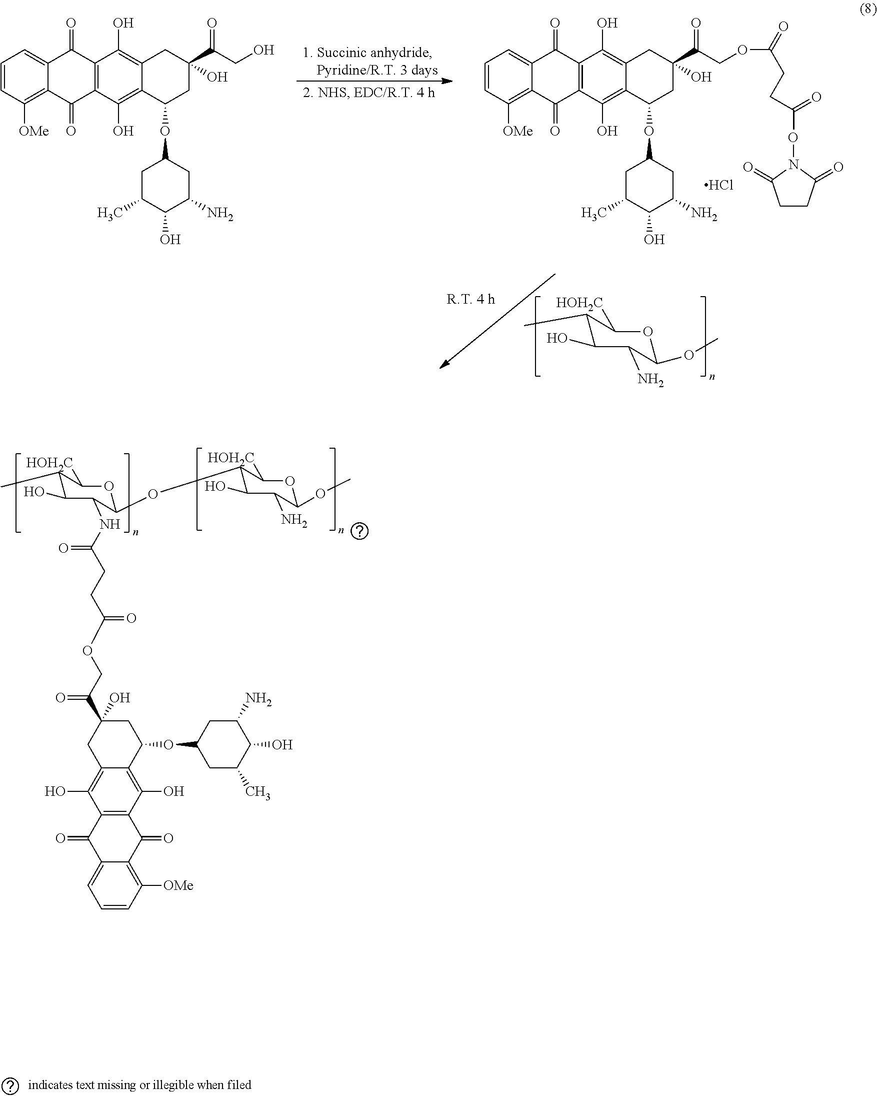

### Experimental Example 13

### Determination of Doxorubicin Content in Doxorubicin-Chitosan Conjugate

In order to determine an amount of doxorubicin present in the doxorubicin-chitosan conjugate prepared above, 0.1 mg of the doxorubicin-chitosan conjugate obtained in Example 12 was dissolved in 1 mL of water and an absorbance was measured on a fluorophotometer at 530 nm (Excitation, 480 nm). The standard curve was plotted by dissolving doxorubicin (1, 5, 10, 15 and 20 μg) in 1 mL of water and measuring the absorbance at the given wavelength. Using the thus-obtained standard curve, the amount of doxorubicin contained in the doxorubicin-chitosan conjugate was calculated. As a result, the content of doxorubicin in the conjugate was determined 15-30% for chitosan of MW 6000.

### VI. Comptothecin-Chitosan Conjugates

### Example 13

### Preparation of Camptothecin Intermediate having Camptothecin Bound to Linker

0.042 g (0.116×19⁻³ mol) of 10-hydroxy camptothecin (JS international, USA) was dissolved in 5 mL of a dichloromethane solution, and 0.015 g (0.152×10⁻³ mol) of succinic anhydride and 18.7×10⁻³ ml (0.232×10⁻³ mol) of pyridine (Sigma) were added to the camptothecin solution. The resulting mixture was stirred at room temperature for 1 days (see Scheme 6). The resulting stirred solution was purified by silica column chromatography and dried to give a camptothecin/succinic acid derivative.

### Example 14

### Construction of Camptothecin-Chitosan Conjugate

0.048 g (0.104×10⁻³ mol) of the camptothecin/succinic acid derivative, 1-ethyl-3-(3-dimethylaminopropyl)carbodiimide hydrochloride (EDC) and N-hydroxysuccinimide (NHS) were dissolved in 3 mL of DMF, and the resulting mixture was stirred at room temperature for 4 hours (see Scheme 9). 0.2 g (66.67×10⁻⁶ mol) of chitosan of MW 6000 (KITTOLIFE, Co., Ltd., Seoul, Korea) was dissolved in a borate buffer solution (3 mL) and DMF (9 mL), which was then added to the above stirred solution and stirred at room temperature for 4 hours (see Scheme 9). The reaction solution was dialyzed against distilled water and freeze-dried to thereby obtain a camptothecin-chitosan conjugate.

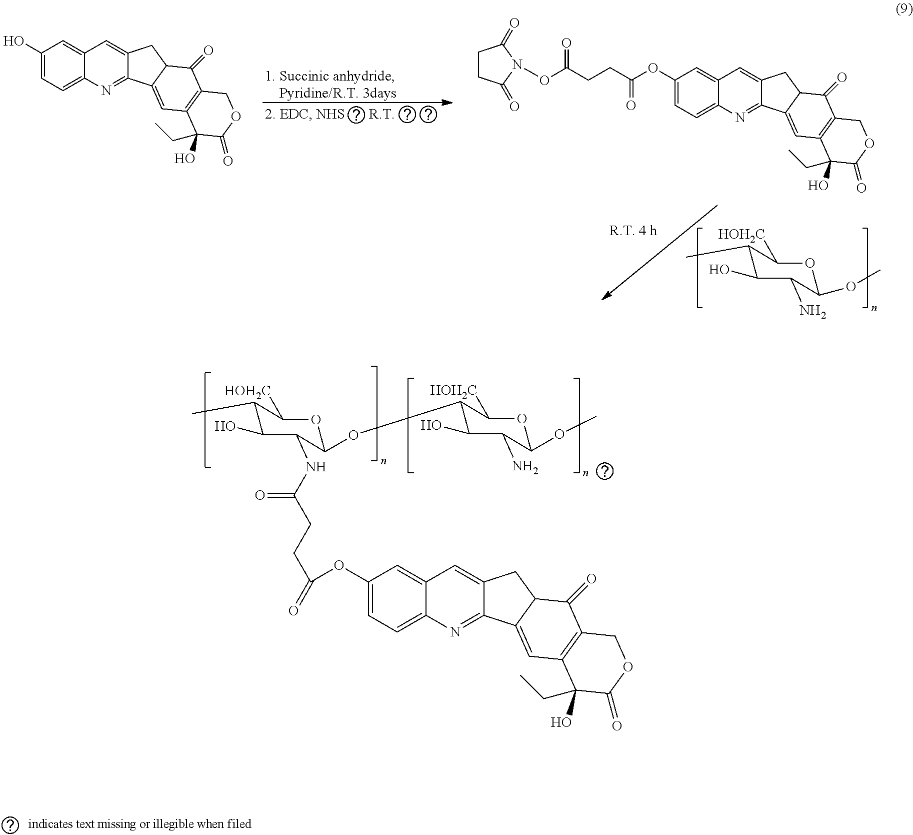

### Experimental Example 14

### Determination of Camptothecin Content in Camptothecin-Chitosan Conjugate

In order to determine an amount of camptothecin present in the camptothecin-chitosan conjugate prepared above, 0.1 mg of the camptothecin-chitosan conjugate obtained in Example 14 was dissolved in 1 mL of acetonitrile/water and an absorbance was measured at a wavelength of UV 365 nm. The standard curve was plotted by dissolving camptothecin (5, 10, 15 and 20 μg) in 1 mL of acetonitrile/water and measuring the absorbance at the given wavelength. Using the thus-obtained standard curve, the amount of camptothecin contained in the camptothecin-chitosan conjugate was calculated. As a result, the content of camptothecin in the conjugate was determined 25-30% for chitosan of MW 6000.

### Experimental Example 15

### Anti-Tumoric Effects by Oral Administration of Anticancer Agent-Chitosan Conjugate

B16F10 melanoma cells Were subcutaneously transplanted at a cell density of 5×10⁶ cells/mice into a dorsal region of CS7BL6 male mice (mean body weight 25 g). When the tumor mass has reached a desired size of about 50 to 100 mm³, animals were divided into a treatment group and a control group. Experiments were carried out for mouse groups, each consisting of 5 to 6 animals having the tumor, simultaneously with observation of changes in tumor. Animals ware given oral administration of the drug or physiological saline for about 30 days, starting on day 10 after tumor transplantation, Bare anticancer agents and anticancer agent-chitosan conjugates were administered to animals at a dose of 25 mg/kg for 5 days, with no administration for following two days. The control group was administered physiological saline or anticancer agents. In order to determine the degree of tumor growth, the size of tumor was daily measured using a calibrator.

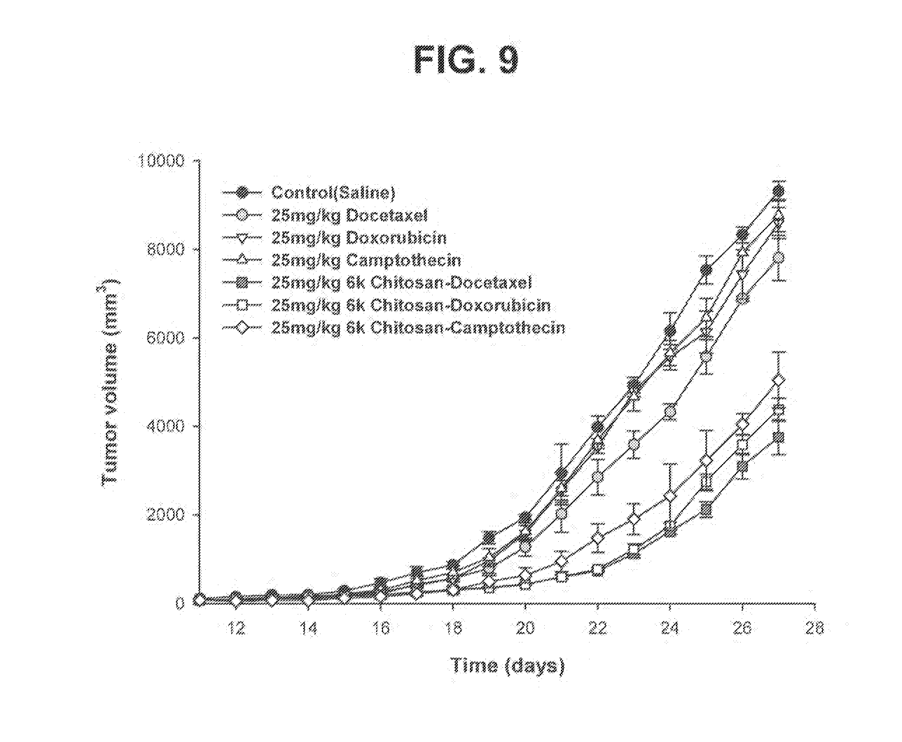

FIG. 9 is a graph showing an anticancer activity in mice with administration of anticancer agents or anticancer agent-chitosan conjugates. The anticancer agent-administered group exhibited no significant difference in the tumor size, as compared to that of the control group. However, it can be seen that the group with the administration of the anticancer agent-chitosan conjugates of the present invention exhibited a significant decrease in the tumor size, as compared to the control group.

Having described a preferred embodiment and other embodiments of the present invention, it is to be understood that variants and modifications thereof falling within the spirit of the invention may become apparent to those skilled in this art, and the scope of the present invention is not limited only to the above-described embodiments; but is elaborated by appended claims and their equivalents.

1-26. (canceled)

27. A method for in vivo delivery of a pharmacologically active substance via a transmucosal route, comprising:
preparing a conjugate by binding covalently the pharmacologically active substance to chitosan or its derivative via a linker; and
administering the conjugate to a subject via the transmucosal route;
wherein the pharmacologically active substance is a protein or peptide

28. (canceled)

29. The method according to claim 1, wherein the pharmacologically active substance is the protein selected from the group consisting of insulin, insulin-like growth factor 1 (IGF-1), growth hormones, interferons (IFNs), erythropoietin, granulocyte-colony stimulating factor (G-CSFs), granulocyte/macrophage-colony stimulating factor (GM-CSFs), interleukin-2 (IL-2), epidermal growth factor (EGF), calcitonin, adrenocorticotropic hormone (ACTH), atobisban, buserelin, cetrorelix, deslorelin, desmopressin, dynorphin A (1-13), elcatonin, eleidosin, eptifibatide, GHRH-II (growth hormone releasing hormone-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclosporin O, exedine, lanreotide, LHRH (luteinizing hormone-releasing hormone), nafarelin, parathyroid hormone, pramlintide, T-20 (enfuvirtide), thymalfasin and ziconotide.

30. The method composition according to claim 29, wherein the protein is insulin or calcitonin.

31-34. (canceled)

35. The method according to claim 27, wherein the chitosan or its derivative has a molecular weight of 500 to 20000 Da.

36. The method according to claim 27, wherein each —NH₂ group of chitosan and the protein or peptide is covalently bound via an amide bond to the linker represented by the following Formula I:
—CO—(CH₂)ₙ—S—S—(CH₂)ₙ—CO— (I)
wherein n is an integer having a value from 1 to 5.

37. (canceled)

38. The method according to claim 27, wherein the conjugate delivers the pharmacologically active substance via buccal, nasal, rectal, vaginal, urethral, throat, alimentary canal, peritoneal or ocular mucosae.

39. The method according to claim 27, wherein the conjugate delivers the pharmacologically active substance via the alimentary canal mucosa.

40. A method for increasing the transmucosal absorption of a pharmacologically active substance of which transmucoal absorption is inhibited by P-glycoprotein, comprising:
preparing a conjugate by binding covalently the pharmacologically active substance to chitosan or its derivative via a linker; and
administering the conjugate to a subject via the transmucosal route;
wherein the pharmacologically active substance is a protein or peptide.

41. (canceled)

42. The method according to claim 40, wherein the pharmacologically active substance is the protein selected from the group consisting of insulin, insulin-like growth factor 1 (IGF-1), growth hormones, interferons (IFNs), erythropoietin, granulocyte-colony stimulating factor (G-CSFs), granulocyte/macrophage-colony stimulating factor (GM-CSFs), interleukin-2 (IL-2), epidermal growth factor (EGF) and calcitonin, adrenocorticotropic hormone (ACTH), atobisban, buserelin, cetrorelix, deslorelin, desmopressin, dynorphin A (1-13), elcatonin, eleidosin, eptifibatide, GHRH-II (growth hormone releasing hormone-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclosporin O, exedine, lanreotide, LHRH (luteinizing hormone-releasing hormone), nafarelin, parathyroid hormone, pramlintide, T-20 (enfuvirtide), thymalfasin and ziconotide.

43. (canceled)

44. The method according to claim 40, wherein the chitosan or its derivative has a molecular weight of 500 to 20000 Da.

45. The method according to claim 8, wherein each —NH₂ group of chitosan and the protein or peptide is covalently bound via an amide bond to the linker represented by the following Formula I:
—CO—(CH2)n-S—S—(CH2)n-CO— (I)
wherein n is an integer of 1 to 5.

46. (canceled)

47. The method according to claim 40, wherein the conjugate delivers the pharmacologically active substance via buccal, nasal, rectal, vaginal, urethral, throat, alimentary canal, peritoneal or ocular mucosae.

48. The method according to claim 40, wherein the conjugate delivers the pharmacologically active substance via the alimentary canal mucosa.
